SEQUENCE LISTING (55 sequences)

SEQ ID NO: 1 - protein, 118 aa, synthetic construct
EVQLVQSGAEVKKPGATVKISCKVSGYSITDYLLHWVQQAPGKGLEWMGWIDPEDAKTKYGQKFQSRVTITADTSTDTAYMELSSLRSEDTAVYYCAANSGTGTMDAWGQGTLVTVSS

SEQ ID NO: 2 - protein, 10 aa, synthetic construct
GYSITDYLLH

SEQ ID NO: 3 - protein, 17 aa, synthetic construct
WIDPEDAKTKYGQKFQS

SEQ ID NO: 4 - protein, 9 aa, synthetic construct
NSGTGTMDA

SEQ ID NO: 5 - protein, 107 aa, synthetic construct
DIVMTQSPATLSVSPGERATLSCKASQNVGSNVDWYQQKPGQSPRVLIYKASIRYTGIPARFSGSGSGTEFTLTISILQSEDFAVYFCMQSKSYPITFGQGTKLEIK

SEQ ID NO: 6 - protein, 11 aa, synthetic construct
KASQNVGSNVD

SEQ ID NO: 7 - protein, 7 aa, synthetic construct
KASIRYT

SEQ ID NO: 8 - protein, 9 aa, synthetic construct
MQSKSYPIT

SEQ ID NO: 9 - protein, 118 aa, synthetic construct
EVQLVQSGAEVKKPGATVKISCKVSGYSITDYLLHWVQQAPGKCLEWMGWIDPEDAKTKYGQKFQSRVTITADTSTDTAYMELSSLRSEDTAVYYCAANSGTGTMDAWGQGTLVTVSS

SEQ ID NO: 10 - protein, 107 aa, synthetic construct
DIVMTQSPATLSVSPGERATLSCKASQNVGSNVDWYQQKPGQSPRVLIYKASIRYTGIPARFSGSGSGTEFTLTISILQSEDFAVYFCMQSKSYPITFGCGTKLEIK

SEQ ID NO: 11 - protein, 123 aa, synthetic construct
EVQLVESGGGLVQPGGSLRLSCAASGSRLDYYAVAWFRQAPGKEREEVSCITYSGGSTNYAESVKGRFTISRDNSKNTLYLQMNSLRAEDTAVYYCTTEPYGSSCPLSGYDTWGQGTLVTVSS

SEQ ID NO: 12 - protein, 10 aa, synthetic construct
GSRLDYYAVA

SEQ ID NO: 13 - protein, 17 aa, synthetic construct
CITYSGGSTNYAESVKG

SEQ ID NO: 14 - protein, 14 aa, synthetic construct
EPYGSSCPLSGYDT

SEQ ID NO: 15 - protein, 117 aa, synthetic construct
QVQLQESGPGLVKPSETLSLTCTVSGSSLTSYGVHWVRQPPGKGLEGLGVIWPGGSTNYNSALMSRVTISKDNSKSQVSLKMSSLTAADTAVYYCARVTGTWYFDVWGQGTTVTVSS

SEQ ID NO: 16 - protein, 10 aa, synthetic construct
GSSLTSYGVH

SEQ ID NO: 17 - protein, 16 aa, synthetic construct
VIWPGGSTNYNSALMS

SEQ ID NO: 18 - protein, 9 aa, synthetic construct
VTGTWYFDV

SEQ ID NO: 19 - protein, 107 aa, synthetic construct
DIQMTQSPSSLSASLGDRVTISCSASQGISNYLNWYQQKPDGTVKLLIYYTSTLHSGVPSRFSGSGSGTDYTLTISSLQPEDIATYYCQQYSKLPWTFGGGTKLEIK

SEQ ID NO: 20 - protein, 11 aa, synthetic construct
SASQGISNYLN

SEQ ID NO: 21 - protein, 7 aa, synthetic construct
YTSTLHS

SEQ ID NO: 22 - protein, 9 aa, synthetic construct
QQYSKLPWT

SEQ ID NO: 23 - protein, 117 aa, synthetic construct
QVQLQESGPGLVKPSETLSLTCTVSGSSLTSYGVHWVRQPPGKCLEGLGVIWPGGSTNYNSALMSRVTISKDNSKSQVSLKMSSLTAADTAVYYCARVTGTWYFDVWGQGTTVTVSS

SEQ ID NO: 24 - protein, 107 aa, synthetic construct
DIQMTQSPSSLSASLGDRVTISCSASQGISNYLNWYQQKPDGTVKLLIYYTSTLHSGVPSRFSGSGSGTDYTLTISSLQPEDIATYYCQQYSKLPWTFGCGTKLEIK

SEQ ID NO: 25 - protein, 107 aa, synthetic construct [sequence omitted]

SEQ ID NO: 26 - protein, 214 aa, synthetic construct
DIQMTQSPSSLSASLGDRVTISCSASQGISNYLNWYQQKPDGTVKLLIYYTSTLHSGVPSRFSGSGSGTDYTLTISSLQPEDIATYYCQQYSKLPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 464 aa, synthetic construct
DIQMTQSPSSLSASLGDRVTISCSASQGISNYLNWYQQKPDGTVKLLIYYTSTLHSGVPSRFSGSGSGTDYTLTISSLQPEDIATYYCQQYSKLPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSEVQLVQSGAEVKKPGATVKISCKVSGYSITDYLLHWVQQAPGKCLEWMGWIDPEDAKTKYGQKFQSRVTITADTSTDTAYMELSSLRSEDTAVYYCAANSGTGTMDAWGQGTLVTVSSGGGGSGGGGSGGGGSDIVMTQSPATLSVSPGERATLSCKASQNVGSNVDWYQQKPGQSPRVLIYKASIRYTGIPARFSGSGSGTEFTLTISILQSEDFAVYFCMQSKSYPITFGCGTKLEIK

SEQ ID NO: 28 - protein, 469 aa, synthetic construct
EVQLVQSGAEVKKPGATVKISCKVSGYSITDYLLHWVQQAPGKCLEWMGWIDPEDAKTKYGQKFQSRVTITADTSTDTAYMELSSLRSEDTAVYYCAANSGTGTMDAWGQGTLVTVSSGGGGSGGGGSGGGGSDIVMTQSPATLSVSPGERATLSCKASQNVGSNVDWYQQKPGQSPRVLIYKASIRYTGIPARFSGSGSGTEFTLTISILQSEDFAVYFCMQSKSYPITFGCGTKLEIKGGGGSGGGGSGGGGSDIQMTQSPSSLSASLGDRVTISCSASQGISNYLNWYQQKPDGTVKLLIYYTSTLHSGVPSRFSGSGSGTDYTLTISSLQPEDIATYYCQQYSKLPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 214 aa, synthetic construct
DIVMTQSPATLSVSPGERATLSCKASQNVGSNVDWYQQKPGQSPRVLIYKASIRYTGIPARFSGSGSGTEFTLTISILQSEDFAVYFCMQSKSYPITFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 463 aa, synthetic construct
DIVMTQSPATLSVSPGERATLSCKASQNVGSNVDWYQQKPGQSPRVLIYKASIRYTGIPARFSGSGSGTEFTLTISILQSEDFAVYFCMQSKSYPITFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSQVQLQESGPGLVKPSETLSLTCTVSGSSLTSYGVHWVRQPPGKCLEGLGVIWPGGSTNYNSALMSRVTISKDNSKSQVSLKMSSLTAADTAVYYCARVTGTWYFDVWGQGTTVTVSSGGGGSGGGGSGGGGSDIQMTQSPSSLSASLGDRVTISCSASQGISNYLNWYQQKPDGTVKLLIYYTSTLHSGVPSRFSGSGSGTDYTLTISSLQPEDIATYYCQQYSKLPWTFGCGTKLEIK

SEQ ID NO: 31 - protein, 468 aa, synthetic construct
QVQLQESGPGLVKPSETLSLTCTVSGSSLTSYGVHWVRQPPGKCLEGLGVIWPGGSTNYNSALMSRVTISKDNSKSQVSLKMSSLTAADTAVYYCARVTGTWYFDVWGQGTTVTVSSGGGGSGGGGSGGGGSDIQMTQSPSSLSASLGDRVTISCSASQGISNYLNWYQQKPDGTVKLLIYYTSTLHSGVPSRFSGSGSGTDYTLTISSLQPEDIATYYCQQYSKLPWTFGCGTKLEIKGGGGSGGGGSGGGGSDIVMTQSPATLSVSPGERATLSCKASQNVGSNVDWYQQKPGQSPRVLIYKASIRYTGIPARFSGSGSGTEFTLTISILQSEDFAVYFCMQSKSYPITFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 347 aa, synthetic construct
DIQMTQSPSSLSASLGDRVTISCSASQGISNYLNWYQQKPDGTVKLLIYYTSTLHSGVPSRFSGSGSGTDYTLTISSLQPEDIATYYCQQYSKLPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSEVQLVESGGGLVQPGGSLRLSCAASGSRLDYYAVAWFRQAPGKEREEVSCITYSGGSTNYAESVKGRFTISRDNSKNTLYLQMNSLRAEDTAVYYCTTEPYGSSCPLSGYDTWGQGTLVTVSS

SEQ ID NO: 33 - protein, 352 aa, synthetic construct
EVQLVESGGGLVQPGGSLRLSCAASGSRLDYYAVAWFRQAPGKEREEVSCITYSGGSTNYAESVKGRFTISRDNSKNTLYLQMNSLRAEDTAVYYCTTEPYGSSCPLSGYDTWGQGTLVTVSSGGGGSGGGGSGGGGSDIQMTQSPSSLSASLGDRVTISCSASQGISNYLNWYQQKPDGTVKLLIYYTSTLHSGVPSRFSGSGSGTDYTLTISSLQPEDIATYYCQQYSKLPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 609 aa, synthetic construct
EVQLVESGGGLVQPGGSLRLSCAASGSRLDYYAVAWFRQAPGKEREEVSCITYSGGSTNYAESVKGRFTISRDNSKNTLYLQMNSLRAEDTAVYYCTTEPYGSSCPLSGYDTWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLQESGPGLVKPSETLSLTCTVSGSSLTSYGVHWVRQPPGKCLEGLGVIWPGGSTNYNSALMSRVTISKDNSKSQVSLKMSSLTAADTAVYYCARVTGTWYFDVWGQGTTVTVSSGGGGSGGGGSGGGGSDIQMTQSPSSLSASLGDRVTISCSASQGISNYLNWYQQKPDGTVKLLIYYTSTLHSGVPSRFSGSGSGTDYTLTISSLQPEDIATYYCQQYSKLPWTFGCGTKLEIK

SEQ ID NO: 35 - protein, 702 aa, synthetic construct
QVQLQESGPGLVKPSETLSLTCTVSGSSLTSYGVHWVRQPPGKGLEGLGVIWPGGSTNYNSALMSRVTISKDNSKSQVSLKMSSLTAADTAVYYCARVTGTWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVQSGAEVKKPGATVKISCKVSGYSITDYLLHWVQQAPGKCLEWMGWIDPEDAKTKYGQKFQSRVTITADTSTDTAYMELSSLRSEDTAVYYCAANSGTGTMDAWGQGTLVTVSSGGGGSGGGGSGGGGSDIVMTQSPATLSVSPGERATLSCKASQNVGSNVDWYQQKPGQSPRVLIYKASIRYTGIPARFSGSGSGTEFTLTISILQSEDFAVYFCMQSKSYPITFGCGTKLEIK

SEQ ID NO: 36 - protein, 447 aa, synthetic construct
QVQLQESGPGLVKPSETLSLTCTVSGSSLTSYGVHWVRQPPGKGLEGLGVIWPGGSTNYNSALMSRVTISKDNSKSQVSLKMSSLTAADTAVYYCARVTGTWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 702 aa, synthetic construct
EVQLVQSGAEVKKPGATVKISCKVSGYSITDYLLHWVQQAPGKCLEWMGWIDPEDAKTKYGQKFQSRVTITADTSTDTAYMELSSLRSEDTAVYYCAANSGTGTMDAWGQGTLVTVSSGGGGSGGGGSGGGGSDIVMTQSPATLSVSPGERATLSCKASQNVGSNVDWYQQKPGQSPRVLIYKASIRYTGIPARFSGSGSGTEFTLTISILQSEDFAVYFCMQSKSYPITFGCGTKLEIKGGGGSGGGGSGGGGSQVQLQESGPGLVKPSETLSLTCTVSGSSLTSYGVHWVRQPPGKGLEGLGVIWPGGSTNYNSALMSRVTISKDNSKSQVSLKMSSLTAADTAVYYCARVTGTWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 702 aa, synthetic construct
EVQLVQSGAEVKKPGATVKISCKVSGYSITDYLLHWVQQAPGKGLEWMGWIDPEDAKTKYGQKFQSRVTITADTSTDTAYMELSSLRSEDTAVYYCAANSGTGTMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLQESGPGLVKPSETLSLTCTVSGSSLTSYGVHWVRQPPGKCLEGLGVIWPGGSTNYNSALMSRVTISKDNSKSQVSLKMSSLTAADTAVYYCARVTGTWYFDVWGQGTTVTVSSGGGGSGGGGSGGGGSDIQMTQSPSSLSASLGDRVTISCSASQGISNYLNWYQQKPDGTVKLLIYYTSTLHSGVPSRFSGSGSGTDYTLTISSLQPEDIATYYCQQYSKLPWTFGCGTKLEIK

SEQ ID NO: 39 - protein, 448 aa, synthetic construct
EVQLVQSGAEVKKPGATVKISCKVSGYSITDYLLHWVQQAPGKGLEWMGWIDPEDAKTKYGQKFQSRVTITADTSTDTAYMELSSLRSEDTAVYYCAANSGTGTMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 702 aa, synthetic construct
QVQLQESGPGLVKPSETLSLTCTVSGSSLTSYGVHWVRQPPGKCLEGLGVIWPGGSTNYNSALMSRVTISKDNSKSQVSLKMSSLTAADTAVYYCARVTGTWYFDVWGQGTTVTVSSGGGGSGGGGSGGGGSDIQMTQSPSSLSASLGDRVTISCSASQGISNYLNWYQQKPDGTVKLLIYYTSTLHSGVPSRFSGSGSGTDYTLTISSLQPEDIATYYCQQYSKLPWTFGCGTKLEIKGGGGSGGGGSGGGGSEVQLVQSGAEVKKPGATVKISCKVSGYSITDYLLHWVQQAPGKGLEWMGWIDPEDAKTKYGQKFQSRVTITADTSTDTAYMELSSLRSEDTAVYYCAANSGTGTMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 585 aa, synthetic construct
QVQLQESGPGLVKPSETLSLTCTVSGSSLTSYGVHWVRQPPGKGLEGLGVIWPGGSTNYNSALMSRVTISKDNSKSQVSLKMSSLTAADTAVYYCARVTGTWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGSRLDYYAVAWFRQAPGKEREEVSCITYSGGSTNYAESVKGRFTISRDNSKNTLYLQMNSLRAEDTAVYYCTTEPYGSSCPLSGYDTWGQGTLVTVSS

SEQ ID NO: 42 - protein, 585 aa, synthetic construct
EVQLVESGGGLVQPGGSLRLSCAASGSRLDYYAVAWFRQAPGKEREEVSCITYSGGSTNYAESVKGRFTISRDNSKNTLYLQMNSLRAEDTAVYYCTTEPYGSSCPLSGYDTWGQGTLVTVSSGGGGSGGGGSGGGGSQVQLQESGPGLVKPSETLSLTCTVSGSSLTSYGVHWVRQPPGKGLEGLGVIWPGGSTNYNSALMSRVTISKDNSKSQVSLKMSSLTAADTAVYYCARVTGTWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 699 aa, synthetic construct
EVQLVQSGAEVKKPGATVKISCKVSGYSITDYLLHWVQQAPGKGLEWMGWIDPEDAKTKYGQKFQSRVTITADTSTDTAYMELSSLRSEDTAVYYCAANSGTGTMDAWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSQVQLQESGPGLVKPSETLSLTCTVSGSSLTSYGVHWVRQPPGKCLEGLGVIWPGGSTNYNSALMSRVTISKDNSKSQVSLKMSSLTAADTAVYYCARVTGTWYFDVWGQGTTVTVSSGGGGSGGGGSGGGGSDIQMTQSPSSLSASLGDRVTISCSASQGISNYLNWYQQKPDGTVKLLIYYTSTLHSGVPSRFSGSGSGTDYTLTISSLQPEDIATYYCQQYSKLPWTFGCGTKLEIK

SEQ ID NO: 44 - protein, 582 aa, synthetic construct
QVQLQESGPGLVKPSETLSLTCTVSGSSLTSYGVHWVRQPPGKGLEGLGVIWPGGSTNYNSALMSRVTISKDNSKSQVSLKMSSLTAADTAVYYCARVTGTWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSEVQLVESGGGLVQPGGSLRLSCAASGSRLDYYAVAWFRQAPGKEREEVSCITYSGGSTNYAESVKGRFTISRDNSKNTLYLQMNSLRAEDTAVYYCTTEPYGSSCPLSGYDTWGQGTLVTVSS

SEQ ID NO: 45 - protein, 582 aa, synthetic construct
EVQLVESGGGLVQPGGSLRLSCAASGSRLDYYAVAWFRQAPGKEREEVSCITYSGGSTNYAESVKGRFTISRDNSKNTLYLQMNSLRAEDTAVYYCTTEPYGSSCPLSGYDTWGQGTLVTVSSGGGGSGGGGSGGGGSQVQLQESGPGLVKPSETLSLTCTVSGSSLTSYGVHWVRQPPGKGLEGLGVIWPGGSTNYNSALMSRVTISKDNSKSQVSLKMSSLTAADTAVYYCARVTGTWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 46 - protein, 330 aa, synthetic construct [sequence omitted]

SEQ ID NO: 47 - protein, 107 aa, synthetic construct [sequence omitted]

SEQ ID NO: 48 - protein, 604 aa, synthetic construct
EVQLVESGGGLVQPGGSLRLSCAASGSRLDYYAVAWFRQAPGKEREEVSCITYSGGSTNYAESVKGRFTISRDNSKNTLYLQMNSLRAEDTAVYYCTTEPYGSSCPLSGYDTWGQGTLVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLQESGPGLVKPSETLSLTCTVSGSSLTSYGVHWVRQPPGKCLEGLGVIWPGGSTNYNSALMSRVTISKDNSKSQVSLKMSSLTAADTAVYYCARVTGTWYFDVWGQGTTVTVSSGGGGSGGGGSGGGGSDIQMTQSPSSLSASLGDRVTISCSASQGISNYLNWYQQKPDGTVKLLIYYTSTLHSGVPSRFSGSGSGTDYTLTISSLQPEDIATYYCQQYSKLPWTFGCGTKLEIK

SEQ ID NO: 49 - protein, 604 aa, synthetic construct
EVQLVESGGGLVQPGGSLRLSCAASGSRLDYYAVAWFRQAPGKEREEVSCITYSGGSTNYAESVKGRFTISRDNSKNTLYLQMNSLRAEDTAVYYCTTEPYGSSCPLSGYDTWGQGTLVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLQESGPGLVKPSETLSLTCTVSGSSLTSYGVHWVRQPPGKGLEGLGVIWPGGSTNYNSALMSRVTISKDNSKSQVSLKMSSLTAADTAVYYCARVTGTWYFDVWGQGTTVTVSSGGGGSGGGGSGGGGSDIQMTQSPSSLSASLGDRVTISCSASQGISNYLNWYQQKPDGTVKLLIYYTSTLHSGVPSRFSGSGSGTDYTLTISSLQPEDIATYYCQQYSKLPWTFGGGTKLEIK

SEQ ID NO: 50 - protein, 609 aa, synthetic construct
EVQLVESGGGLVQPGGSLRLSCAASGSRLDYYAVAWFRQAPGKEREEVSCITYSGGSTNYAESVKGRFTISRDNSKNTLYLQMNSLRAEDTAVYYCTTEPYGSSCPLSGYDTWGQGTLVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLQESGPGLVKPSETLSLTCTVSGSSLTSYGVHWVRQPPGKCLEGLGVIWPGGSTNYNSALMSRVTISKDNSKSQVSLKMSSLTAADTAVYYCARVTGTWYFDVWGQGTTVTVSSGGGGSGGGGSGGGGSGGGGSDIQMTQSPSSLSASLGDRVTISCSASQGISNYLNWYQQKPDGTVKLLIYYTSTLHSGVPSRFSGSGSGTDYTLTISSLQPEDIATYYCQQYSKLPWTFGCGTKLEIK

SEQ ID NO: 51 - protein, 609 aa, synthetic construct
EVQLVESGGGLVQPGGSLRLSCAASGSRLDYYAVAWFRQAPGKEREEVSCITYSGGSTNYAESVKGRFTISRDNSKNTLYLQMNSLRAEDTAVYYCTTEPYGSSCPLSGYDTWGQGTLVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLQESGPGLVKPSETLSLTCTVSGSSLTSYGVHWVRQPPGKGLEGLGVIWPGGSTNYNSALMSRVTISKDNSKSQVSLKMSSLTAADTAVYYCARVTGTWYFDVWGQGTTVTVSSGGGGSGGGGSGGGGSGGGGSDIQMTQSPSSLSASLGDRVTISCSASQGISNYLNWYQQKPDGTVKLLIYYTSTLHSGVPSRFSGSGSGTDYTLTISSLQPEDIATYYCQQYSKLPWTFGGGTKLEIK

SEQ ID NO: 52 - protein, 609 aa, synthetic construct
EVQLVESGGGLVQPGGSLRLSCAASGSRLDYYAVAWFRQAPGKEREEVSCITYSGGSTNYAESVKGRFTISRDNSKNTLYLQMNSLRAEDTAVYYCTTEPYGSSCPLSGYDTWGQGTLVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLQESGPGLVKPSETLSLTCTVSGSSLTSYGVHWVRQPPGKGLEGLGVIWPGGSTNYNSALMSRVTISKDNSKSQVSLKMSSLTAADTAVYYCARVTGTWYFDVWGQGTTVTVSSGGGGSGGGGSGGGGSDIQMTQSPSSLSASLGDRVTISCSASQGISNYLNWYQQKPDGTVKLLIYYTSTLHSGVPSRFSGSGSGTDYTLTISSLQPEDIATYYCQQYSKLPWTFGGGTKLEIK

SEQ ID NO: 53 - protein, 615 aa, synthetic construct
EVQLVESGGGLVQPGGSLRLSCAASGSRLDYYAVAWFRQAPGKEREEVSCITYSGGSTNYAESVKGRFTISRDNSKNTLYLQMNSLRAEDTAVYYCTTEPYGSSCPLSGYDTWGQGTLVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLQESGPGLVKPSETLSLTCTVSGSSLTSYGVHWVRQPPGKGLEGLGVIWPGGSTNYNSALMSRVTISKDNSKSQVSLKMSSLTAADTAVYYCARVTGTWYFDVWGQGTTVTVSSSPNSASHSGSAPQTSSAPGSQDIQMTQSPSSLSASLGDRVTISCSASQGISNYLNWYQQKPDGTVKLLIYYTSTLHSGVPSRFSGSGSGTDYTLTISSLQPEDIATYYCQQYSKLPWTFGGGTKLEIK

SEQ ID NO: 54 - protein, 601 aa, synthetic construct
EVQLVESGGGLVQPGGSLRLSCAASGSRLDYYAVAWFRQAPGKEREEVSCITYSGGSTNYAESVKGRFTISRDNSKNTLYLQMNSLRAEDTAVYYCTTEPYGSSCPLSGYDTWGQGTLVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLQESGPGLVKPSETLSLTCTVSGSSLTSYGVHWVRQPPGKGLEGLGVIWPGGSTNYNSALMSRVTISKDNSKSQVSLKMSSLTAADTAVYYCARVTGTWYFDVWGQGTTVTVSSGGGSAAADIQMTQSPSSLSASLGDRVTISCSASQGISNYLNWYQQKPDGTVKLLIYYTSTLHSGVPSRFSGSGSGTDYTLTISSLQPEDIATYYCQQYSKLPWTFGGGTKLEIK

SEQ ID NO: 55 - protein, 614 aa, synthetic construct
EVQLVESGGGLVQPGGSLRLSCAASGSRLDYYAVAWFRQAPGKEREEVSCITYSGGSTNYAESVKGRFTISRDNSKNTLYLQMNSLRAEDTAVYYCTTEPYGSSCPLSGYDTWGQGTLVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLQESGPGLVKPSETLSLTCTVSGSSLTSYGVHWVRQPPGKCLEGLGVIWPGGSTNYNSALMSRVTISKDNSKSQVSLKMSSLTAADTAVYYCARVTGTWYFDVWGQGTTVTVSSGGGGSGGGGSGGGGSGGGGSDIQMTQSPSSLSASLGDRVTISCSASQGISNYLNWYQQKPDGTVKLLIYYTSTLHSGVPSRFSGSGSGTDYTLTISSLQPEDIATYYCQQYSKLPWTFGCGTKLEIK

(19) United States
(12) Patent Application Publication
(10) Pub. No.: US 2026/0049151 A1
(43) Pub. Date: Feb. 19, 2026

(54) BISPECIFIC ANTIBODY BINDING TO TNFR2 AND 4-1BB

(71) Applicant: SHENGHE (CHINA) BIOPHARMACEUTICAL CO., LTD., Nanjing, Jiangsu (CN)

(72) Inventors: Jinhua ZHOU, Nanjing (CN); Chongbing WU, Nanjing (CN); Xiaoling JIANG, Nanjing (CN); Liusong YIN, Nanjing (CN)

(73) Assignee: SHENGHE (CHINA) BIOPHARMACEUTICAL CO., LTD., Nanjing, Jiangsu (CN)

(21) Appl. No.: 19/102,873

(22) PCT Filed: Aug. 18, 2023
(86) PCT No.: PCT/CN2023/113758

§ 371 Date: Feb. 11, 2025

(30) Foreign Application Priority Data
Aug. 19, 2022 (WO) PCT/CN2022/113459

(51) Int. Cl. C07K 16/28; A61K 38/00; C07K 16/46

(52) U.S. Cl. CPC C07K 16/2878; A61K 38/00; C07K 16/468; C07K 2317/31; C07K 2317/51; C07K 2317/515; C07K 2317/569; C07K 2317/622; C07K 2317/75; C07K 2317/76

(57) ABSTRACT

A bispecific antibody, including: a first antibody or antigen-binding fragment thereof that specifically binds to a first antigen; a second antibody or antigen-binding fragment thereof that specifically binds to a second antigen; where the first antigen is TNFR2, and the second antigen is 4-1BB; or, the first antigen is 4-1BB, and the second antigen is TNFR2. The bispecific antibody is a T cell regulator of the tumor microenvironment, which can stimulate the proliferation of CD8+T cells, activate CD8+T cells, and release anti-tumor factors, such as IFNγ and IL2 by using a TNFR2 agonistic antibody, or can block TNF-TNFR2 and inhibit the proliferation of Treg by using a TNFR2 antagonistic antibody. The bispecific antibody can simultaneously target 4-1BB, activate the 4-1BB signaling pathway of T cells and NK cells, enhance the killing power of cytotoxic T cells and NK cells, and produce anti-tumor activity.

### TECHNICAL FIELD

The present invention belongs to the field of biomedicine, and specifically relates to a bispecific antibody that binds to TNFR2 and 4-1BB and use thereof.

### BACKGROUND

Tumor necrosis factor receptor 2 (TNFR2), also known as tumor necrosis factor receptor superfamily 1B (TNFRSF1B) and CD120b, is a 75-kDa type I transmembrane protein that belongs to the tumor necrosis factor receptor superfamily member, which contains an extracellular domain (ECD, residues 1-257) and an intracellular domain (ICD, residues 288-461) with a TRAF2-binding domain. In normal T cells, TNFA-TNFR2 interaction triggers cell survival signals through the NFKB signaling pathway. However, in autoimmune T cells, TNFA-TNFR2 interaction triggers apoptosis signals through the caspase pathway.

Currently, TNFR2 has been shown to enhance the activation of effector T cells (Teff) and reduce the suppression mediated by regulatory T cells (Treg). TNFR2 activation induces signal transduction through the mitogen-activated protein kinase (MAPK) signaling pathway, which coordinates the transcription of genes that promote escape from apoptosis and cell proliferation mediated by TRAF2/3 signaling and NFKB. TNFR2 can be expressed not only on cancer cells and tumor-infiltrating Tregs, but also on effector Teff cells. Studies have shown that TNFR2 agonist monoclonal antibodies can stimulate the proliferation of CD8+T cells, activate CD8+ T cells, and release anti-tumor factors such as IFNγ and IL2; TNFR2 antagonist monoclonal antibodies can block TNF-TNFR2 and inhibit Treg proliferation.

The body's immune response process requires the participation of multiple immune cells and immune molecules, among which the activation of T cells is the core of the immune response. Generally, two different signals are required for T cells to be fully activated: the first signal is generated by the combination of TCR expressed by T cells and antigens presented by the major histocompatibility complex (MHC). The second is various stimulation signals that activate or turn off the switch of T cell function. PD-L1 is a ligand that turns off T cell function. Among the ligands that activate T cell function, 4-1BBL (ligand of 4-1BB) is an important member.

4-1BB is a T cell co-stimulatory molecule and an important member of the tumor necrosis factor receptor (TNF) superfamily. It is mainly expressed in activated CD4+ and CD8+T cells, and natural killer cells (NK cells). 4-1BBL is mainly expressed in activated antigen presenting cells (APCs), such as monocytes, phagocytes, dendritic cells (DC cells), B cells, etc. The interaction between 4-1BB and its ligand can provide a second signal for T cell activation independent of the CD28 signal. When the two are combined, the co-stimulatory signal can promote the proliferation and activation of T cells and inhibit activation-induced apoptosis (a major type of programmed cell death of T cells, referred to as AICD), thereby enhancing the immune killing function of T cells. At the same time, it induces the activation of monocytes, dendritic cells, etc., promotes the secretion of cytokines, and enables these cells to play a role in immune regulation. Therefore, the signaling pathway related to 4-1BB is involved in the immune regulation process of diseases such as autoimmune diseases, tumors, viral infections, transplant rejection, and inflammation. 4-1BB is also considered to be a very potential target for enhancing anti-tumor immunity.

### SUMMARY

In this application, the inventors have developed a bispecific antibody with good performance that can bind to TNFR2 and 4-1BB. The bispecific antibody of the present invention is a T cell regulator of the tumor microenvironment, which can use agonistic anti-TNFR2 monoclonal antibodies to stimulate the proliferation of CD8+T cells, activate CD8+T cells, and release anti-tumor factors such as IFNγ and IL2; TNFR2 antagonists block TNF-TNFR2 and inhibit Treg proliferation. The bispecific antibody of the present invention can simultaneously target 4-1BB, activate the 4-1BB signaling pathway of T cells and NK cells, enhance the killing power of cytotoxic T cells and NK cells, and produce anti-tumor activity.

The present invention provides a bispecific antibody that specifically binds to TNFR2 and 4-1BB, comprising: (a) a first antibody or antigen-binding fragment thereof that specifically binds to a first antigen; and (b) a second antibody or antigen-binding fragment thereof that specifically binds to a second antigen; wherein the first antigen is TNFR2, and the second antigen is 4-1BB; or, the first antigen is 4-1BB, and the second antigen is TNFR2.

In some embodiments, the first antibody or antigen-binding fragment thereof comprises a heavy chain and a light chain; and the second antibody or antigen-binding fragment thereof comprises scFv or VHH.

In some embodiments, the heavy chain variable region of one heavy chain and the light chain variable region of one light chain of the first antibody form an antigen-binding site, and the heavy chain variable region of another heavy chain and the light chain variable region of another light chain form an antigen-binding site.

In some embodiments, the bispecific antibody comprises one first antibody or antigen-binding fragment thereof and one or more scFv.

In some embodiments, the bispecific antibody comprises one first antibody or antigen-binding fragment thereof and one scFv, wherein the scFv is connected to the N-terminus of the heavy chain or light chain of the first antibody or antigen-binding fragment thereof.

In some embodiments, the bispecific antibody comprises one first antibody or antigen-binding fragment thereof and one scFv, wherein the scFv is connected to the C-terminus of the heavy chain or light chain of the first antibody or antigen-binding fragment thereof.

In some embodiments, the bispecific antibody comprises a first antibody or antigen-binding fragment thereof and the two scFvs.

In some embodiments, the two scFvs are respectively connected to the N-terminus of two heavy chains of the first antibody or antigen-binding fragment thereof.

In some embodiments, the two scFvs are respectively connected to the N-terminus of two light chains of the first antibody or antigen-binding fragment thereof.

In some embodiments, the two scFvs are respectively connected to the C-terminus of two heavy chains of the first antibody or antigen-binding fragment thereof.

In some embodiments, the two scFvs are respectively connected to the C-terminus of two light chains of the first antibody or antigen-binding fragment thereof.

In some embodiments, the bispecific antibody comprises one first antibody that specifically binds to TNFR2 and two scFvs that specifically bind to 4-1BB, wherein the two scFvs that specifically bind to 4-1BB are respectively connected to the N-terminus of two heavy chains of the first antibody.

In some embodiments, the bispecific antibody comprises one first antibody that specifically binds to TNFR2 and two scFvs that specifically bind to 4-1BB, wherein the two scFvs that specifically bind to 4-1BB are respectively connected to the C-terminus of two heavy chains of the first antibody.

In some embodiments, the bispecific antibody comprises one first antibody that specifically binds to TNFR2 and two scFvs that specifically bind to 4-1BB, wherein the two scFvs that specifically bind to 4-1BB are respectively connected to the N-terminus of two light chains of the first antibody.

In some embodiments, the bispecific antibody comprises one first antibody that specifically binds to TNFR2 and two scFvs that specifically bind to 4-1BB, wherein the two scFvs that specifically bind to 4-1BB are respectively connected to the C-terminus of two light chains of the first antibody.

In some embodiments, the bispecific antibody comprises one first antibody that specifically binds to 4-1BB and two scFvs that specifically bind to TNFR2, wherein the two scFvs that specifically bind to TNFR2 are respectively connected to the N-terminus of two heavy chains of the first antibody.

In some embodiments, the bispecific antibody comprises one first antibody that specifically binds to 4-1BB and two scFvs that specifically bind to TNFR2, wherein the two scFvs that specifically bind to TNFR2 are respectively connected to the C-terminus of two heavy chains of the first antibody.

In some embodiments, the bispecific antibody comprises one first antibody that specifically binds to 4-1BB and two scFvs that specifically bind to TNFR2, wherein the two scFvs that specifically bind to TNFR2 are respectively connected to the N-terminus of two light chains of the first antibody.

In some embodiments, the bispecific antibody comprises one first antibody that specifically binds to 4-1BB and two scFvs that specifically bind to TNFR2, wherein the two scFvs that specifically bind to TNFR2 are respectively connected to the C-terminus of two light chains of the first antibody.

In some embodiments, the bispecific antibody comprises two first polypeptide chains and two second polypeptide chains, and for each of the polypeptide chains: (a) the first polypeptide chain independently comprises the light chain of the first antibody or antigen-binding fragment thereof; and (b) the second polypeptide chain independently comprises the heavy chain of the first antibody or antigen-binding fragment thereof and the scFv.

In some embodiments, the bispecific antibody comprises two first polypeptide chains and two second polypeptide chains, for each of the polypeptide chains: (a) the first polypeptide chain independently comprises the light chain of the first antibody or antigen-binding fragment thereof and the scFv; and (b) the second polypeptide chain independently comprises the heavy chain of the first antibody or antigen-binding fragment thereof.

In some embodiments, the two first polypeptide chains of the bispecific antibody are identical or different, and/or the two second polypeptide chains are identical or different.

In some embodiments, the bispecific antibody comprises one first antibody or antigen-binding fragment thereof and one or more VHH.

In some embodiments, the bispecific antibody comprises one first antibody or antigen-binding fragment thereof and one VHH, wherein the VHH is connected to the N-terminus of the heavy chain or light chain of the first antibody or antigen-binding fragment thereof.

In some embodiments, the bispecific antibody comprises one first antibody or antigen-binding fragment thereof and one VHH, wherein the VHH is connected to the C-terminus of the heavy chain or light chain of the first antibody or antigen-binding fragment thereof.

In some embodiments, the bispecific antibody comprises one first antibody or antigen-binding fragment thereof and two VHHs.

In some embodiments, the two VHHs are respectively connected to the N-terminus of two heavy chains of the first antibody or antigen-binding fragment thereof.

In some embodiments, the two VHHs are respectively connected to the N-terminus of two light chains of the first antibody or antigen-binding fragment thereof.

In some embodiments, the two VHHs are respectively connected to the C-terminus of two heavy chains of the first antibody or antigen-binding fragment thereof.

In some embodiments, the two VHHs are respectively connected to the C-terminus of two light chains of the first antibody or antigen-binding fragment thereof.

In some embodiments, the bispecific antibody comprises one first antibody that specifically binds to 4-1BB and two VHHs that specifically bind to TNFR2, wherein the two VHHs that specifically bind to TNFR2 are respectively connected to the N-terminus of two heavy chains of the first antibody.

In some embodiments, the bispecific antibody comprises one first antibody that specifically binds to 4-1BB and two VHHs that specifically bind to TNFR2, wherein the two VHHs that specifically bind to TNFR2 are respectively connected to the C-terminus of two heavy chains of the first antibody.

In some embodiments, the bispecific antibody comprises one first antibody that specifically binds to 4-1BB and two VHHs that specifically bind to TNFR2, wherein the two VHHs that specifically bind to TNFR2 are respectively connected to the N-terminus of two light chains of the first antibody.

In some embodiments, the bispecific antibody comprises one first antibody that specifically binds to 4-1BB and two VHHs that specifically bind to TNFR2, wherein the two VHHs that specifically bind to TNFR2 are respectively connected to the C-terminus of two light chains of the first antibody.

In some embodiments, the bispecific antibody comprises two first polypeptide chains and two second polypeptide chains, and for each of the polypeptide chains: (a) the first polypeptide chain independently comprises the light chain of the first antibody or antigen-binding fragment thereof; and (b) the second polypeptide chain independently comprises the heavy chain of the first antibody or antigen-binding fragment thereof and the VHH.

In some embodiments, the bispecific antibody comprises two first polypeptide chains and two second polypeptide chains, and for each of the polypeptide chains: (a) the first polypeptide chain independently comprises the light chain of the first antibody or antigen-binding fragment thereof and the VHH; and (b) the second polypeptide chain independently comprises the heavy chain of the first antibody or antigen-binding fragment thereof.

In some embodiments, the two first polypeptide chains of the bispecific antibody are identical or different, and/or the two second polypeptide chains are identical or different.

In some embodiments, the heavy chain variable region and the light chain variable region of the scFv are connected by a linker L1.

In some embodiments, the scFv is connected to the N-terminus or C-terminus of the heavy chain or light chain of the first antibody or antigen-binding fragment thereof by a linker L2.

In some embodiments, the linker L1 and linker L2 are identical or different.

In some embodiments, the linker L1 and/or linker L2 has an amino acid sequence as shown in (G₄S)ₓ, where x is an integer selected from 1-6; preferably, the linker L1 and/or linker L2 is (G₄S)₂, (G₄S)₃ or (G₄S)₄.

In some embodiments, the heavy chain of the first antibody or antigen-binding fragment thereof comprises a heavy chain variable region and a heavy chain constant region, and the light chain comprises a light chain variable region and a light chain constant region; preferably, the first antibody or antigen-binding fragment thereof is a full-length antibody.

In some embodiments, the heavy chain of the first antibody or antigen-binding fragment thereof comprises a first Fc region and a second Fc region. In some embodiments, the first Fc region and the second Fc region are identical or different. In some embodiments, the Fc region is selected from IgG, IgA, IgD, IgE, IgM and variants thereof. In some embodiments, the Fc region is selected from IgG1, IgG2, IgG3, IgG4 and variants thereof. In some embodiments, the Fc region comprises one or more amino acid mutations, preferably amino acid substitutions, insertions or deletions.

In some embodiments, the first antibody or antigen-binding fragment thereof specifically binds to 4-1BB, wherein HCDR1 of the first antibody or antigen-binding fragment thereof is as shown in SEQ ID NO: 16, or is a sequence having at least 80% identity to SEQ ID NO: 16; HCDR2 is as shown in SEQ ID NO: 17, or is a sequence having at least 80% identity to SEQ ID NO: 17; HCDR3 is as shown in SEQ ID NO: 18, or is a sequence having at least 80% identity to SEQ ID NO: 18; LCDR1 is as shown in SEQ ID NO: 20, or is a sequence having at least 80% identity to SEQ ID NO: 20; LCDR2 is as shown in SEQ ID NO: 21, or is a sequence having at least 80% identity to SEQ ID NO: 21; and LCDR3 is as shown in SEQ ID NO: 22, or is a sequence having at least 80% identity to SEQ ID NO: 22.

In some embodiments, the scFv specifically binds to TNFR2, and HCDR1 of the scFv is as shown in SEQ ID NO: 2, or is a sequence having at least 80% identity to SEQ ID NO: 2; HCDR2 is as shown in SEQ ID NO: 3, or is a sequence having at least 80% identity to SEQ ID NO: 3; HCDR3 is as shown in SEQ ID NO: 4, or is a sequence having at least 80% identity to SEQ ID NO: 4; LCDR1 is as shown in SEQ ID NO: 6, or is a sequence having at least 80% identity to SEQ ID NO: 6; LCDR2 is as shown in SEQ ID NO: 7, or is a sequence having at least 80% identity to SEQ ID NO: 7; and LCDR3 is as shown in SEQ ID NO: 8, or is a sequence having at least 80% identity to SEQ ID NO: 8.

In some embodiments, the VHH specifically binds to TNFR2, and HCDR1 of the VHH is as shown in SEQ ID NO: 12, or is a sequence having at least 80% identity to SEQ ID NO: 12; HCDR2 is as shown in SEQ ID NO: 13, or is a sequence having at least 80% identity to SEQ ID NO: 13; and HCDR3 is as shown in SEQ ID NO: 14, or is a sequence having at least 80% identity to SEQ ID NO: 14.

In some embodiments, the first antibody or antigen-binding fragment thereof specifically binds to 4-1BB, wherein HCDR1 of the first antibody or antigen-binding fragment thereof is as shown in SEQ ID NO: 16; HCDR2 is as shown in SEQ ID NO: 17; HCDR3 is as shown in SEQ ID NO: 18; LCDR1 is as shown in SEQ ID NO: 20; LCDR2 is as shown in SEQ ID NO: 21; and LCDR3 is as shown in SEQ ID NO: 22. The scFv specifically binds to TNFR2, wherein HCDR1 of the scFv is as shown in SEQ ID NO: 2; HCDR2 is as shown in SEQ ID NO: 3; HCDR3 is as shown in SEQ ID NO: 4; LCDR1 is as shown in SEQ ID NO: 6; LCDR2 is as shown in SEQ ID NO: 7; and LCDR3 is as shown in SEQ ID NO: 8.

In some embodiments, the first antibody or antigen-binding fragment thereof specifically binds to 4-1BB, wherein HCDR1 of the first antibody or antigen-binding fragment thereof is as shown in SEQ ID NO: 16; HCDR2 is as shown in SEQ ID NO: 17; HCDR3 is as shown in SEQ ID NO: 18; LCDR1 is as shown in SEQ ID NO: 20; LCDR2 is as shown in SEQ ID NO: 21; and LCDR3 is as shown in SEQ ID NO: 22. The VHH specifically binds to TNFR2, wherein HCDR1 of the VHH is as shown in SEQ ID NO: 12; HCDR2 is as shown in SEQ ID NO: 13; and HCDR3 is as shown in SEQ ID NO: 14.

In some embodiments, the first antibody or antigen-binding fragment thereof specifically binds to TNFR2, wherein HCDR1 of the first antibody or antigen-binding fragment thereof is as shown in SEQ ID NO: 2, or is a sequence having at least 80% identity to SEQ ID NO: 2; HCDR2 is as shown in SEQ ID NO: 3, or is a sequence having at least 80% identity to SEQ ID NO: 3; HCDR3 is as shown in SEQ ID NO: 4, or is a sequence having at least 80% identity to SEQ ID NO: 4; LCDR1 is as shown in SEQ ID NO: 6, or is a sequence having at least 80% identity to SEQ ID NO: 6; LCDR2 is as shown in SEQ ID NO: 7, or is a sequence having at least 80% identity to SEQ ID NO: 7; and LCDR3 is as shown in SEQ ID NO: 8, or is a sequence having at least 80% identity to SEQ ID NO: 8.

In some embodiments, the scFv specifically binds to 4-1BB, wherein HCDR1 of the scFv is as shown in SEQ ID NO: 16, or is a sequence having at least 80% identity to SEQ ID NO: 16; HCDR2 is as shown in SEQ ID NO: 17, or is a sequence having at least 80% identity to SEQ ID NO: 17; HCDR3 is as shown in SEQ ID NO: 18, or is a sequence having at least 80% identity to SEQ ID NO: 18; LCDR1 is as shown in SEQ ID NO: 20, or is a sequence having at least 80% identity to SEQ ID NO: 20; LCDR2 is as shown in SEQ ID NO: 21, or is a sequence having at least 80% identity to SEQ ID NO: 21; and LCDR3 is as shown in SEQ ID NO: 22, or is a sequence having at least 80% identity to SEQ ID NO: 22.

In some embodiments, the first antibody or antigen-binding fragment thereof specifically binds to TNFR2, wherein HCDR1 of the first antibody or antigen-binding fragment thereof is as shown in SEQ ID NO: 2; HCDR2 is as shown in SEQ ID NO: 3; HCDR3 is as shown in SEQ ID NO: 4; LCDR1 is as shown in SEQ ID NO: 6; LCDR2 is as shown in SEQ ID NO: 7; and LCDR3 is as shown in SEQ ID NO: 8. The scFv specifically binds to 4-1BB, wherein HCDR1 of the scFv is as shown in SEQ ID NO: 16; HCDR2 is as shown in SEQ ID NO: 17; HCDR3 is as shown in SEQ ID NO: 18; LCDR1 is as shown in SEQ ID NO: 20; LCDR2 is as shown in SEQ ID NO: 21; and LCDR3 is as shown in SEQ ID NO: 22.

In some embodiments, the first antibody or antigen-binding fragment thereof specifically binds to 4-1BB, wherein the heavy chain variable region VH of the first antibody or antigen-binding fragment thereof is as shown in SEQ ID NO: 15, or is a sequence having at least 80% identity to SEQ ID NO: 15; and the light chain variable region VL is as shown in SEQ ID NO: 19, or is a sequence having at least 80% identity to SEQ ID NO: 19.

In some embodiments, the scFv specifically binds to TNFR2, wherein the heavy chain variable region VH of the scFv is as shown in SEQ ID NO: 9, or is a sequence having at least 80% identity to SEQ ID NO: 9; and the light chain variable region VL is as shown in SEQ ID NO: 10, or is a sequence having at least 80% identity to SEQ ID NO: 10.

In some embodiments, the VHH specifically binds to TNFR2, wherein the heavy chain variable region VH of the VHH is as shown in SEQ ID NO: 11, or is a sequence having at least 80% identity to SEQ ID NO: 11.

In some embodiments, the first antibody or antigen-binding fragment thereof specifically binds to TNFR2, wherein the heavy chain variable region VH of the first antibody or antigen-binding fragment thereof is as shown in SEQ ID NO: 1, or is a sequence having at least 80% identity to SEQ ID NO: 1; and the light chain variable region VL is as shown in SEQ ID NO: 5, or is a sequence having at least 80% identity to SEQ ID NO: 5.

In some embodiments, the scFv specifically binds to 4-1BB, wherein the heavy chain variable region VH of the scFv is as shown in SEQ ID NO: 23, or is a sequence having at least 80% identity to SEQ ID NO: 23; and the light chain variable region VL is as shown in SEQ ID NO: 24, or is a sequence having at least 80% identity to SEQ ID NO: 24.

In some embodiments, the first polypeptide chain of the bispecific antibody is selected from any one of SEQ ID NOs: 26-33, or an amino acid sequence having at least 80% identity to any one of SEQ ID NOs: 26-33; and the second polypeptide chain of the bispecific antibody is selected from any one of SEQ ID NOs: 35-45, or an amino acid sequence having at least 80% identity to any one of SEQ ID NOs: 35-45.

In some embodiments, the bispecific antibody comprises:
(1) the first polypeptide chain as shown in SEQ ID NO: 26, and the second polypeptide chain as shown in SEQ ID NO: 35;
(2) the first polypeptide chain as shown in SEQ ID NO: 27, and the second polypeptide chain as shown in SEQ ID NO: 36;
(3) the first polypeptide chain as shown in SEQ ID NO: 26, and the second polypeptide chain as shown in SEQ ID NO: 37;
(4) the first polypeptide chain as shown in SEQ ID NO: 28, and the second polypeptide chain as shown in SEQ ID NO: 36;
(5) the first polypeptide chain as shown in SEQ ID NO: 29, and the second polypeptide chain as shown in SEQ ID NO: 38;
(6) the first polypeptide chain as shown in SEQ ID NO: 29, and the second polypeptide chain as shown in SEQ ID NO: 43;
(7) the first polypeptide chain as shown in SEQ ID NO: 30, and the second polypeptide chain as shown in SEQ ID NO: 39;
(8) the first polypeptide chain as shown in SEQ ID NO: 29, and the second polypeptide chain as shown in SEQ ID NO: NO: 40;
(9) the first polypeptide chain as shown in SEQ ID NO: 31, and the second polypeptide chain as shown in SEQ ID NO: 39;
(10) the first polypeptide chain as shown in SEQ ID NO: 26, and the second polypeptide chain as shown in SEQ ID NO: 41;
(11) the first polypeptide chain as shown in SEQ ID NO: 26, and the second polypeptide chain as shown in SEQ ID NO: 44;
(12) the first polypeptide chain as shown in SEQ ID NO: 32, and the second polypeptide chain as shown in SEQ ID NO: 36;
(13) the first polypeptide chain as shown in SEQ ID NO: 26, and the second polypeptide chain as shown in SEQ ID NO: 42;
(14) the first polypeptide chain as shown in SEQ ID NO: 26, and the second polypeptide chain as shown in SEQ ID NO: 45; or
(15) the first polypeptide chain as shown in SEQ ID NO: 33, and the second polypeptide chain as shown in SEQ ID NO: 36.

The present invention also provides a bispecific antibody that can specifically bind to TNFR2 and 4-1BB, comprising: (a) a first antibody or antigen-binding fragment thereof that specifically binds to a first antigen; and (b) a second antibody or antigen-binding fragment thereof that specifically binds to a second antigen; wherein the first antigen is TNFR2 and the second antigen is 4-1BB.

In some embodiments, the first antibody or antigen-binding fragment thereof comprises a heavy chain and a light chain; and the second antibody or antigen-binding fragment thereof comprises a scFv.

In some embodiments, the Fab region of the first antibody or antigen-binding fragment thereof is replaced with a VHH that specifically binds to TNFR2.

In some embodiments, it comprises one first antibody or antigen-binding fragment thereof and one or more scFv.

In some embodiments, it comprises one first antibody or antigen-binding fragment thereof and one scFv, wherein the scFv is connected to the C-terminus of the heavy chain of the first antibody or antigen-binding fragment thereof.

In some embodiments, it comprises one first antibody or antigen-binding fragment thereof and the two scFvs.

In some embodiments, the two scFvs are respectively connected to the C-terminus of two heavy chains of the first antibody or antigen-binding fragment thereof.

In some embodiments, the bispecific antibody comprises two polypeptide chains, wherein each of the polypeptide chains independently comprises the first antibody or antigen-binding fragment thereof and the scFv.

In some embodiments, the two polypeptide chains are identical or different.

In some embodiments, the scFv is connected to the C-terminus of the heavy chain of the first antibody or antigen-binding fragment thereof by a linker L3.

In some embodiments, the linker L3 has an amino acid sequence as shown in (G₄S)ₓ, where x is an integer selected from 1-6; preferably, the linker L1 and/or linker L2 is (G₄S)₂, (G₄S)₃ or (G₄S)₄.

In some embodiments, the heavy chain of the first antibody or antigen-binding fragment thereof comprises a first Fc region and a second Fc region. In some embodiments, the first Fc region and the second Fc region are identical or different. In some embodiments, the Fc region is selected from IgG, IgA, IgD, IgE and/or IgM. In some embodiments, the Fc region is selected from IgG1, IgG2, IgG3 and/or IgG4. In some embodiments, the Fc region comprises one or more amino acid mutations, preferably amino acid substitutions, insertions or deletions.

In some embodiments, the VHH that specifically binds to TNFR2, wherein HCDR1 of the VHH is as shown in SEQ ID NO: 12, or is a sequence having at least 80% identity to SEQ ID NO: 12; HCDR2 is as shown in SEQ ID NO: 13, or is a sequence having at least 80% identity to SEQ ID NO: 13; and HCDR3 is as shown in SEQ ID NO: 14, or is a sequence having at least 80% identity to SEQ ID NO: 14.

In some embodiments, the scFv specifically binds to 4-1BB, and HCDR1 of the scFv is as shown in SEQ ID NO: 16, or is a sequence having at least 80% identity to SEQ ID NO: 16; HCDR2 is as shown in SEQ ID NO: 17, or is a sequence having at least 80% identity to SEQ ID NO: 17; HCDR3 is as shown in SEQ ID NO: 18, or is a sequence having at least 80% identity to SEQ ID NO: 18; LCDR1 is as shown in SEQ ID NO: 20, or is a sequence having at least 80% identity to SEQ ID NO: 20; LCDR2 is as shown in SEQ ID NO: 21, or is a sequence having at least 80% identity to SEQ ID NO: 21; LCDR3 is as shown in SEQ ID NO: 22, or is a sequence having at least 80% identity to SEQ ID NO: 22.

In some embodiments, the heavy chain variable region VH of the VHH that specifically binds to TNFR2 is as shown in SEQ ID NO: 11, or is a sequence having at least 80% identity to SEQ ID NO: 11.

In some embodiments, the scFv specifically binds to 4-1BB, and the heavy chain variable region VH of the scFv is as shown in SEQ ID NO: 23, or is a sequence having at least 80% identity to SEQ ID NO: 23; and the light chain variable region VL is as shown in SEQ ID NO: 24, or is a sequence having at least 80% identity to SEQ ID NO: 24.

In some embodiments, the polypeptide chain of the bispecific antibody is selected from any one of the amino acid sequences of SEQ ID NOs: 34, 48-55, or is a sequence having at least 80% identity to any one of the amino acid sequences of SEQ ID NOs: 34, 48-55.

The present invention provides an isolated nucleic acid molecule, comprising a nucleotide sequence encoding the bispecific antibody described in any of the foregoing. Preferably, the isolated nucleic acid molecule comprises a nucleotide sequence encoding a polypeptide chain of the bispecific antibody described in any of the foregoing.

The present invention provides a multifunctional fusion protein, comprising the bispecific antibody described in any of the foregoing.

In some embodiments, the multifunctional fusion protein further comprises one or more third antibodies or antigen-binding portions thereof that specifically bind to other antigens.

In some embodiments, the antigen that binds to the third antibody or antigen-binding portion thereof is selected from a tumor-associated antigen (TAA) or an immune checkpoint.

In some embodiments, the antigen that binds to the third antibody or its antigen-binding portion is selected from GPC3, CD19, CD20 (MS4A1), CD22, CD30, CD33, CD38, CD40, CD123, CD133, CD138, CDK4, CEA, Claudin18.2, AFP, ALK, BAGE protein, BCMA, BIRC5 (survivin), BIRC7, β-catenin, brc-ab1, BRCA1, BORIS, CA9, CA125, carbon Anhydrase IX, caspase-8, CALR, CCR5, NA17, NKG2D, NY-BR1, NY-BR62, NY-BR85, NY-ESO1, OX40, p15, p53, PAP, PAX3, PAX5, PCTA-1, PLACI, PRLR, PRAME, PSMA (FOLH1), RAGE proteins, cyclin-B1, CYP1B1, EGFR, EGFRVIII, ErbB2/Her2, ErbB3, ErbB4, ETV6-AM L, EpCAM, EphA2, Fra-1, FOLR1, GAGE protein, GD2, GD3, GloboH, GM3, gp100, Her2, HLA/B-raf, HLA/k-ras, HLA/MAGE-A3, hTERT, I L13Ra2, LMP2, κ-Light, LeY, MAGE-1, MAGE-2, MAGE-3, MAGE-4, MAGE-6, MAGE-12, MART-1, mesothelin, ML-IAP, MOv-γ, Muc1, Muc2, Mu c3, Muc4, Muc5, Muc16, MUM1, Ras, RGS5, Rho, ROR1, SART-1, SART-3, STEAP1, STEAP2, TAG-72, TGF-β, TMPRSS2, Tang-Novo antigen, TRP-1, TRP-2, tyrosinase and urokinin-3, 5T4, PD-L1, CTLA4, PD-L2, PD-1, CD47, TIGIT, GITR, TIM3, ILT4, TREM2, LAG3, CD27, CD24, B7H3, or B7H4.

In some embodiments, the multifunctional fusion protein further comprises a cytokine. In some embodiments, the cytokine is selected from the group consisting of IL-1, IL-2, IL-2 Rα, IL-2 Rβ, IL-3, IL-3 Rα, IL-4, IL-4 Rα, IL-5, IL-5 Rα, IL-6, IL-6 Rα, IL-7, IL-7 Rα, IL-8, IL-9, IL-9 Rα, IL-10, IL-10R1, IL-10R2, IL-11, IL-11Rα, IL-12, IL-12 Rα, IL-12 Rβ2, IL-12 Rβ1, IL-13, IL-13 Rα, IL-13 Rα2, IL-14, IL-15, IL-15Rα sushi, IL-16, IL-17, IL-18, IL-19, IL-20, IL-20R1, IL-20R2, IL-21, IL-21 Ra, IL-22, IL-23, IL-23R, IL-27R, IL-31R, TGF, VEGF, IFNγ, IFNα, or GM-CSF.

The present invention also provides use of the bispecific antibody described in any of the foregoing or the multifunctional fusion protein described in any of the foregoing in the preparation of a drug for the treatment of cancer.

In some embodiments, the cancer is selected from the group consisting of human brain astroblastoma, human pharyngeal cancer, adrenal tumor, AIDS-related cancer, alveolar soft tissue sarcoma, astrocytoma, bladder cancer, bone cancer, brain and spinal cord cancer, metastatic brain tumor, breast cancer, carotid body tumor, cervical cancer, chondrosarcoma, chordoma, renal chromophobe cell carcinoma, clear cell carcinoma, colon cancer, colorectal cancer, desmoplastic small round cell tumor, ependymoma, Ewing tumor, extraskeletal myxoid chondrosarcoma, fibrous dysplasia, fibrous dysplasia, gallbladder or bile duct cancer, gastric cancer, gestational trophoblastic disease, germ cell tumor, head and neck cancer, hepatocellular carcinoma, pancreatic islet cell tumor, Kaposi's sarcoma, kidney cancer, leukemia, liposarcoma/malignant lipomatous tumor, liver cancer, lymphoma, lung cancer, medulloblastoma, melanoma, meningioma, multiple endocrine neoplasia, multiple myeloma, myelodysplastic syndrome, neuroblastoma, neuroendocrine tumors, ovarian cancer, pancreatic cancer, papillary thyroid cancer, parathyroid tumor, pediatric cancer, peripheral nerve sheath tumor, pheochromocytoma, pituitary tumor, prostate cancer, posterior uveal melanoma, metastatic kidney cancer, rhabdoid tumor, rhabdomyosarcoma, sarcoma, skin cancer, soft tissue sarcoma, squamous cell carcinoma, synovial sarcoma, testicular cancer, thymic cancer, thymoma, metastatic thyroid cancer, or uterine cancer.

The present invention also provides use of the bispecific antibody described in any of the foregoing or the multifunctional fusion protein described in any of the foregoing in the preparation of a drug for the treatment autoimmune disease.

In some embodiments, the autoimmune disease is selected from graft-versus-host disease, rheumatoid arthritis, Crohn's disease, multiple sclerosis, colitis, psoriasis, autoimmune uveitis, pemphigus, epidermolysis bullosa, or type I diabetes.

In some embodiments, the use is achieved by one or more of tumor immunotherapy, cell therapy, or gene therapy.

The present invention also provides a pharmaceutical composition, comprising the bispecific antibody described in any of the foregoing and a pharmaceutically acceptable carrier, diluent or excipient.

The present invention also provides a pharmaceutical composition, comprising the multifunctional fusion protein described in any of the foregoing and a pharmaceutically acceptable carrier, diluent or excipient.

The present invention also provides an antibody-drug conjugate, comprising the bispecific antibody described in any of the foregoing.

In some embodiments, the conjugated drug is selected from a cytotoxic agent, a small molecule chemical agent, or an immunotoxin.

### Abbreviations and Definitions

The following abbreviations are used herein: VH: antibody heavy chain variable region; VL: antibody light chain variable region; CDR: complementarity determinusng region in immunoglobulin variable region; FR: antibody framework region, i.e., amino acid residues in antibody variable region other than CDR residues; IgG: immunoglobulin G.

The term “antibody” refers to a natural immunoglobulin or an immunoglobulin prepared by partial or complete synthesis. Antibodies can be reconstructed and separated from natural resources such as plasma or serum in which the antibody is naturally present, or from the culture supernatant of hybridoma cells that produce the antibody, from animal immune serum, or from phage library screening. Alternatively, it can be partially or completely synthesized using techniques such as gene recombination. Preferred antibodies include, for example, antibodies of isotypes of immunoglobulins or subclasses of these isotypes. Known human immunoglobulins include 9 categories (isotypes) of IgG1, IgG2, IgG3, IgG4, IgA1, IgA2, IgD, IgE, and IgM. Among these isotypes, antibodies of the present invention can include IgG1, IgG2, IgG3, and/or IgG4.

The term “chimeric antibody” refers to an antibody formed by fusing the variable region of a mouse antibody with the constant region of a human antibody, which can reduce the immune response induced by the mouse antibody. To establish a chimeric antibody, it is necessary to first establish a hybridoma that secretes mouse-specific monoclonal antibodies, then clone the variable region gene from the mouse hybridoma cells, and then clone the constant region gene of the human antibody as needed, connect the mouse variable region gene with the human constant region gene into a chimeric gene and insert it into a human vector, and finally express the chimeric antibody molecule in a eukaryotic or prokaryotic industrial system.

Some antibodies used in this article are immunoglobulin molecules composed of two pairs of polypeptide chains, each pair having a light chain (LC) and a heavy chain (HC). Each heavy chain consists of a heavy chain variable region (VH) and a heavy chain constant region (CH). The heavy chain constant region consists of 3 domains (CH1, CH2 and CH3). Each light chain consists of a light chain variable region (VL) and a light chain constant region (CL), or only a light chain constant region (CL). The light chain constant region consists of one domain CL. The constant domain is not directly involved in the binding of antibodies to antigens, but exhibits a variety of effector functions, such as mediating the binding of immunoglobulins to host tissues or factors, including various cells of the immune system (e.g., effector cells) and the first component (C1q) of the classical complement system. The VH and VL regions can also be subdivided into regions with high variability (called complementarity determinusng regions (CDRs)), interspersed with more conservative regions called framework regions (FRs). Each VH and VL consists of three CDRs and four FRs arranged from the amino terminus to the carboxyl terminus in the following order: FR1, CDR1, FR2, CDR2, FR3, CDR3, FR4. The variable regions (VH and VL) of each heavy chain/light chain pair form the antigen binding site.

The term “bispecific antibody” refers to a protein molecule that can specifically bind to two target antigens or target antigen epitopes. In the present invention, “bispecific antigen-binding protein” comprising an antibody or antigen-binding fragment (such as Fab, scFv, etc.) and “bispecific antibody” can be used interchangeably.

The term “antigen-binding fragment” of an antibody refers to a polypeptide fragment of an antibody, such as a polypeptide fragment of a full-length antibody, which retains the ability to specifically bind to the same antigen to which the full-length antibody binds, and/or competes with the full-length antibody for specific binding to the antigen, which is also referred to as an “antigen-binding portion”. Antigen-binding fragments of antibodies can be produced by recombinant DNA technology or by enzymatic or chemical cleavage of intact antibodies. Non-limiting examples of antigen-binding fragments include Fab, Fab′, F(ab′)2, Fd, Fv, dAb, and complementary determinusng region (CDR) fragments, single-chain antibodies (e.g., scFv), chimeric antibodies, diabodies, linear antibodies, nanobodies (such as technology from Ablynx), domain antibodies (such as technology from Domantis), and polypeptides that contain at least a portion of an antibody sufficient to confer specific antigen-binding ability to a polypeptide.

The term “antibody drug conjugate” or “ADC” refers to a binding protein (such as an antibody or antigen-binding fragment thereof) connected to one or more conjugated drugs (which may optionally be a therapeutic agent or a cytotoxic agent), and its structure generally consists of three parts: an antibody or antibody-like ligand, a drug portion, and a linker that couples the antibody or antibody-like ligand and the drug. ADCs generally have 1, 2, 3, 4, 5, 6, 7, 8, 9, or 10 drugs conjugated to the antibody.

The term “polypeptide” refers to an amino acid chain of any length, regardless of modification (e.g., phosphorylation or glycosylation). The term polypeptide includes proteins and fragments thereof. Polypeptides can be “exogenous,” meaning that they are “heterologous,” i.e., foreign to the host cell utilized, such as human polypeptides produced by bacterial cells. Polypeptides are disclosed herein as amino acid residue sequences. Those sequences are written from left to right in the direction of the amino terminus to the carboxyl terminus. Amino acid residue sequences are named by three-letter or one-letter codes according to standard nomenclature.

The term “scFv” refers to a molecule comprising an antibody heavy chain variable domain (VH) and an antibody light chain variable domain (VL) connected by a linker. Such scFv molecules may have a general structure: NH2-VL-linker-VH-COOH or NH2-VH-linker-VL-COOH. Suitable prior art linkers consist of repeated GGGGS amino acid sequences or variants thereof, for example using 1-6 repeated GGGGS amino acid sequences or variants thereof.

The term “VHH” refers to a single antigen-binding polypeptide comprising only one heavy chain variable region (VHH), which is derived from the variable domain of a heavy chain molecule that is naturally free of light chains, to distinguish it from the conventional VH of a four-chain immunoglobulin. Such VHH molecules can be derived from antibodies produced in camelid species such as camels, llamas, llamas, dromedaries, alpacas and guanacos. Other species outside the camelidae family can produce heavy chain molecules that naturally lack light chains, and such VHH are within the scope of this application.

The term “Fab” consists of a complete L chain and the variable region domain (VH) of the H chain and the first constant domain (CH1) of one heavy chain. Each Fab fragment is monovalent for antigen binding, i.e., it has a single antigen binding site. For example, Fab fragments can be produced recombinantly or by papain digestion of full-length antibodies.

The term “host cell” refers to a cell that has been or can be transformed with a nucleic acid sequence and thereby expresses a selected target gene. The term includes the offspring of a parent cell, whether or not the offspring is identical to the original parent cell in morphology or genetic composition, as long as the offspring has the selected target gene. Commonly used host cells include bacteria, yeast, mammalian cells, etc.

The term “vector” refers to a nucleic acid molecule capable of propagating another nucleic acid to which it is connected. The term includes vectors that are self-replicating nucleic acid structures as well as vectors that are incorporated into the genome of a host cell into which they are introduced. Certain vectors are capable of directing the expression of nucleic acids to which they are operably linked and are referred to herein as “expression vectors.”

The term “pharmaceutically acceptable carrier” includes any of the standard pharmaceutical carriers, such as phosphate-buffered saline solutions, water, and emulsions, as well as various types of wetting agents.

The term “identity” is defined as the percentage of amino acid residues in a candidate sequence that are identical to the amino acid residues in a reference polypeptide sequence, after aligning the sequences and introducing gaps, if necessary, to obtain the maximum percentage sequence identity. Comparisons for purposes of determinusng percentage amino acid sequence identity can be performed in a variety of ways within the skill of the art, for example using publicly available computer software such as BLAST software or FASTA packages.

The term “at least 80% identity” means that the percentage of amino acid residues in the candidate sequence that are identical to the amino acid residues in the reference polypeptide sequence is greater than 80%, including 80%, 81%, 82%, 83%, 84%, 85%, 86%, 87%, 88%, 89%, 90%, 91%, 92%, 93%, 94%, 95%, 96%, 97%, 98%, 99%, and 100%.

There are a variety of methods/systems in the art to define and describe CDRs, and these systems and/or definitions have been developed and refined over the years, including Kabat, Chothia, IMGT, AbM, and Contact. Kabat is the most commonly used, defining CDRs based on sequence variability; Chothia defines CDRs based on sequence variability based on the position of the structural loop region; the IMGT system defines CDRs based on sequence variability and position within the variable domain structure; AbM is defined based on Oxford Molecular's AbM antibody modeling software, which is a compromise between Kabat and Chothia; Contact defines CDRs based on the analysis of complex crystal structures, which is similar to Chothia in many aspects. The CDRs of this application are mainly divided using Kabat, and some CDRs are divided using definition standards, such as the HCDR1 shown in SEQ ID NO: 2, 12, and 16, which starts at the fourth position after the first cysteine in the heavy chain variable region, and the length of HCDR1 is generally 10-12, ending at the amino acid before tryptophan.

The term “specificity” means that one of the molecules involved in specific binding does not show any significant binding to molecules other than one or more of the binding partner molecules. In addition, the term is also used when the domain containing the antibody variable region is specific for a particular epitope among multiple epitopes in an antigen. When the epitope bound by the domain containing the antibody variable region is contained in several different antigens, the antigen-binding molecule containing the domain containing the antibody variable region can bind to various antigens having the epitope.

The term “epitope” refers to an antigenic determinant in an antigen, and refers to an antigenic site bound by a domain of an antigen binding molecule comprising an antibody variable region disclosed in this specification. Therefore, an epitope can be defined based on its structure. In addition, the epitope can also be defined based on the antigen binding activity in an antigen binding molecule that recognizes the epitope. When the antigen is a peptide or polypeptide, the epitope can be specified by the amino acid residues that form the epitope; when the epitope is a sugar chain, the epitope can be determined by its specific sugar chain structure.

The term “positive control” refers to a natural or engineered cell or antibody that can bind to or express a target protein. The positive control referred to herein refers to a single-target positive control.

The term “negative control” refers to the use of the same species, subtype, dose, immunoglobulin and subtype of immunoglobulin, and the same marker as the experimental sample in the same experiment to eliminate the experimental background effect of non-specific binding samples on the experimental values, as a control to better illustrate the experimental effect.

The terms “Treg”, “Treg cells” or “regulatory T cells” are sometimes referred to as suppressor T cells, characterized by the expression of the biomarkers CD4, FOXP3 and CD25, and represent a T cell subset that regulates the immune system, maintains tolerance to self-antigens and prevents autoimmune diseases. Tregs are immunosuppressive and generally inhibit or downregulate the induction and proliferation of effector T (Teff) cells. Tregs can develop in the thymus (so-called CD4+Foxp3+ “natural” Tregs) or differentiate from naive CD4+ T cells in the periphery, for example, after exposure to TGFβ or retinoic acid.

The terms “Teff”, “Teff cells” or “effector T cells” refer to cells formed after T cells are stimulated by antigens, proliferate and differentiate. Effector T cells have the function of releasing lymphokines, and in this process, a small number of T cells become memory T cells. Effector T cells contact target cells and stimulate granule exocytosis. The released perforins form small holes on the surface of target cells through polymerization, thereby mediating the killing effect. The target cell death process is similar to apoptosis. At the same time, effector T cells can also release immune active substances-lymphokines, such as interleukins, interferons, etc.

### BRIEF DESCRIPTION OF THE DRAWINGS

FIG. 1 shows an exemplary bispecific antibody comprising a full-length antibody which specifically recognizes 4-1BB and a scFv which specifically recognizes TNFR2, wherein the scFv is connected to the C-terminus of two heavy chains of the full-length antibody by a linker.

FIG. 2 shows an exemplary bispecific antibody comprising a full-length antibody which specifically recognizes 4-1BB and a scFv which specifically recognizes TNFR2, wherein the scFv is connected to the C-terminus of two light chains of the full-length antibody by a linker.

FIG. 3 shows an exemplary bispecific antibody comprising a full-length antibody which specifically recognizes 4-1BB and a scFv which specifically recognizes TNFR2, wherein the scFv is connected to the N-terminus of two heavy chains of the full-length antibody by a linker.

FIG. 4 shows an exemplary bispecific antibody comprising a full-length antibody which specifically recognizes 4-1BB and a scFv which specifically recognizes TNFR2, wherein the scFv is connected to the N-terminus of two light chains of the full-length antibody by a linker.

FIG. 5 shows an exemplary bispecific antibody comprising a full-length antibody which specifically recognizes TNFR2 and a scFv which specifically recognizes 4-1BB, wherein the scFv is connected to the C-terminus of two heavy chains of the full-length antibody by a linker.

FIG. 6 shows an exemplary bispecific antibody comprising a full-length antibody which specifically recognizes TNFR2 and a scFv which specifically recognizes 4-1BB, wherein the scFv is connected to the C-terminus of two light chains of the full-length antibody by a linker.

FIG. 7 shows an exemplary bispecific antibody comprising a full-length antibody which specifically recognizes TNFR2 and a scFv which specifically recognizes 4-1BB, wherein the scFv is connected to the N-terminus of two heavy chains of the full-length antibody by a linker.

FIG. 8 shows an exemplary bispecific antibody comprising a full-length antibody which specifically recognizes TNFR2 and a scFv which specifically recognizes 4-1BB, wherein the scFv is connected to the N-terminus of two light chains of the full-length antibody by a linker.

FIG. 9 shows an exemplary bispecific antibody comprising a full-length antibody which specifically recognizes 4-1BB and a VHH which specifically recognizes TNFR2, wherein the VHH is connected to the C-terminus of two heavy chains of the full-length antibody by a linker.

FIG. 10 shows an exemplary bispecific antibody comprising a full-length antibody which specifically recognizes 4-1BB and a VHH which specifically recognizes TNFR2, wherein the VHH is connected to the C-terminus of two light chains of the full-length antibody by a linker.

FIG. 11 shows an exemplary bispecific antibody comprising a full-length antibody which specifically recognizes 4-1BB and a VHH which specifically recognizes TNFR2, wherein the VHH is connected to the N-terminus of two heavy chains of the full-length antibody by a linker.

FIG. 12 shows an exemplary bispecific antibody comprising a full-length antibody which specifically recognizes 4-1BB and a VHH which specifically recognizes TNFR2, wherein the VHH is connected to the N-terminus of two light chains of the full-length antibody by a linker.

FIG. 13 shows an exemplary bispecific antibody, wherein the Fab region of the bispecific antibody is replaced by a VHH which specifically recognizes TNFR2, and further comprises a scFv which specifically recognizes 4-1BB, wherein the scFv is connected to the C-terminus of two heavy chains of the antibody by a linker.

FIG. 14 shows the binding activity of antibodies A, B and C to TNFR2 protein.

FIG. 15 shows the binding activity of antibodies D, E1 and F to TNFR2 protein.

FIG. 16 shows the binding activity of antibodies G and H to TNFR2 protein.

FIG. 17 shows the binding activity of antibodies J1 and K to TNFR2 protein.

FIG. 18 shows the binding activity of antibodies M1 and N to TNFR2 protein.

FIG. 19 shows the binding activity of antibodies E1 and E2 to TNFR2 protein.

FIG. 20 shows the binding activity of antibodies J2 and M2 to TNFR2 protein.

FIG. 21 shows the binding activity of antibody O to TNFR2 protein.

FIG. 22 shows the binding activity of antibodies A, B and C to 4-1BB protein.

FIG. 23 shows the binding activity of antibodies D, E1 and F to 4-1BB protein.

FIG. 24 shows the binding activity of antibodies G, H and J1 to 4-1BB protein.

FIG. 25 shows the binding activity of antibodies K, M1 and N to 4-1BB protein.

FIG. 26 shows the binding activity of antibodies E1, E2 and J2 to 4-1BB protein.

FIG. 27 shows the binding activity of antibody O to 4-1BB protein.

FIG. 28 shows the dual-terminal binding activities of antibodies A, B, C and D.

FIG. 29 shows the dual-terminal binding activities of antibodies E1, F, G and H.

FIG. 30 shows the dual-terminal binding activities of antibodies J1, K, M1 and N.

FIG. 31 shows the dual-terminal binding activities of antibodies A, B, C and D.

FIG. 32 shows the dual-terminal binding activities of antibodies E1, F, G and H.

FIG. 33 shows the dual-terminal binding activities of antibodies J1, K, M1 and N.

FIG. 34 shows the activation effects of antibodies E1, H, J1 and M1 on HEK-293/NFκB-Luci/4-1BB cells.

FIG. 35 shows the activation effect of antibodies O and J1 on HEK-293/NFκB-Luci/4-1BB cells.

FIG. 36 shows the anti-tumor growth curves of antibodies E1, E2, J2, M2 and O.

FIG. 37 shows the binding activity of antibodies O1, O2, O3 and O4 to TNFR2 protein.

FIG. 38 shows the binding activity of antibodies O1, O5 and O6 to TNFR2 protein.

FIG. 39 shows the binding activity of antibodies O1, O7 and O8 to TNFR2 protein.

FIG. 40 shows the binding activity of antibodies O1, O2, O3 and O4 to 4-1BB protein.

FIG. 41 shows the binding activity of antibodies O1, O5 and O6 to 4-1BB protein.

FIG. 42 shows the binding activity of antibodies O1, O7 and O8 to 4-1BB protein.

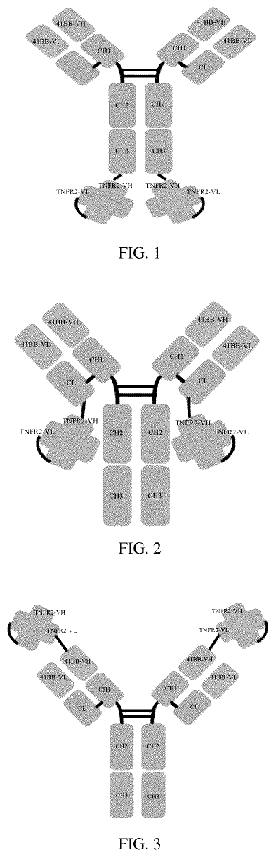

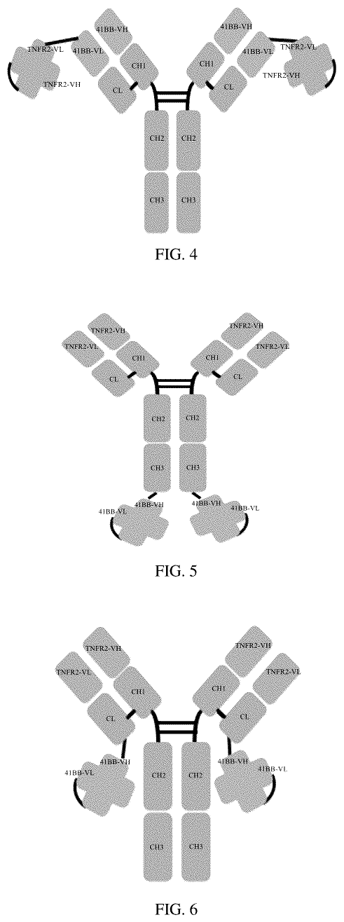

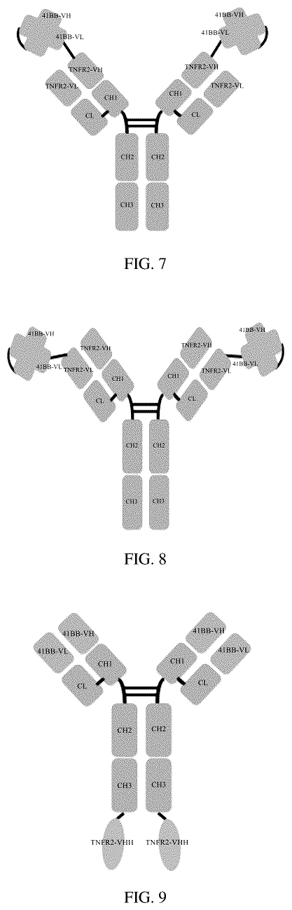

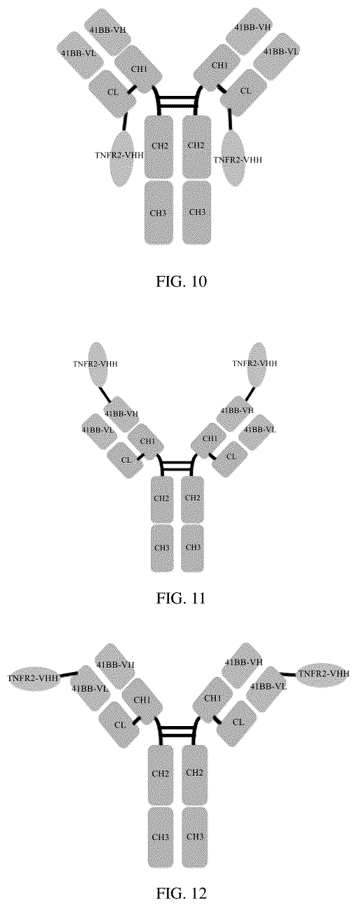

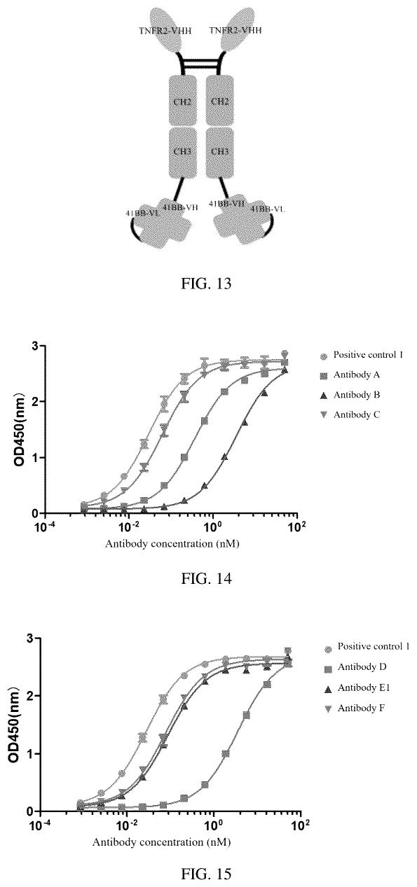

### DETAILED DESCRIPTION

The present invention is further described below in conjunction with the accompanying drawings and specific examples, and the protection content of the present invention is not limited to the following examples. It should also be understood that the terms used in the examples of the present invention are to describe specific specific embodiments, rather than to limit the scope of protection of the present invention. Without departing from the spirit and scope of the inventive concept, changes and advantages that can be thought of by those skilled in the art are all included in the present invention, and the attached claims and any equivalents thereof are the scope of protection of the present invention.

### Example 1 Preparation of Bispecific Antibodies with Different Structures

The sequences of the TNFR2 antibody as a full-length antibody, scFv, and VHH, and the sequences of the 4-1BB antibody as a full-length antibody and scFv are shown in Table 1.

TABLE 1
TNFR2 antibody and 4-1BB antibody sequence list

| | TNFR2 | | | 4-1BB | |
|---|---|---|---|---|---|
| | Sequence 1 (full | Sequence 2 | Sequence 3 | Sequence 4 (full | Sequence 5 |
| | length antibody) | (scFv) | (VHH) | length antibody) | (scFv) |
| VH | SEQ ID NO: 1 | SEQ ID NO: 9 | SEQ ID NO: 11 | SEQ ID NO: 15 | SEQ ID NO: 23 |
| HCDR1 | SEQ ID NO: 2 | SEQ ID NO: 2 | SEQ ID NO: 12 | SEQ ID NO: 16 | SEQ ID NO: 16 |
| HCDR2 | SEQ ID NO: 3 | SEQ ID NO: 3 | SEQ ID NO: 13 | SEQ ID NO: 17 | SEQ ID NO: 17 |
| HCDR3 | SEQ ID NO: 4 | SEQ ID NO: 4 | SEQ ID NO: 14 | SEQ ID NO: 18 | SEQ ID NO: 18 |
| VL | SEQ ID NO: 5 | SEQ ID NO: 10 | / | SEQ ID NO: 19 | SEQ ID NO: 24 |
| LCDR1 | SEQ ID NO: 6 | SEQ ID NO: 6 | / | SEQ ID NO: 20 | SEQ ID NO: 20 |
| LCDR2 | SEQ ID NO: 7 | SEQ ID NO: 7 | / | SEQ ID NO: 21 | SEQ ID NO: 21 |
| LCDR3 | SEQ ID NO: 8 | SEQ ID NO: 8 | / | SEQ ID NO: 22 | SEQ ID NO: 22 |

The sequence of the TNFR2 antibody and the sequence of the 4-1BB antibody are connected in a certain order through a linker to form a peptide chain in Table 2. In this embodiment, the linker used in the bispecific antibody has 2 GGGGS repeats (i.e., GGGGSGGGGS, hereinafter abbreviated as (G₄S)₂) or 3 GGGGS repeats (i.e., GGGGSGGGGSGGGGS, hereinafter abbreviated as (G₄S)₃), and the CL used in this embodiment is of kappa (κ) type, and the sequence is as shown in SEQ ID NO: 25.

TABLE 2
Peptide chain sequence list

| Peptide | | Amino acid | |
|---|---|---|---|
| chain | Structural Order | sequence | Remark |
| L1 | VL(4-1BB)-CL(kappa) | SEQ ID NO: 26 | / |
| L2 | VL(4-1BB)-CL(kappa)-(G₄S)₂-VH(TNFR2)- | SEQ ID NO: 27 | / |
| | (G₄S)₃-VL(TNFR2) | | |
| L3 | VH(TNFR2)-(G₄S)₃-VL(TNFR2)-(G₄S)₃-VL | SEQ ID NO: 28 | / |
| | (4-1BB)-CL(kappa) | | |
| L4 | VL(TNFR2)-CL(kappa) | SEQ ID NO: 29 | / |
| L5 | VL(TNFR2)-CL(kappa)-(G₄S)₂-VH(4-1BB)- | SEQ ID NO: 30 | / |
| | (G₄S)₃-VL(4-1BB) | | |
| L6 | VH(4-1BB)-(G₄S)₃-VL(4-1BB)-(G₄S)₃- | SEQ ID NO: 31 | / |
| | VL(TNFR2))-CL(kappa) | | |
| L7 | VL(4-1BB)-CL(kappa)-(G₄S)₂-VHH(TNFR2) | SEQ ID NO: 32 | / |
| L8 | VHH(TNFR2)-(G₄S)₃-VL(4-1BB)-CL(kappa) | SEQ ID NO: 33 | / |
| H1 | VHH(TNFR2)-Hinge-CH2-CH3-(G₄S)₃-VH | SEQ ID NO: 34 | IgG1 subtype |
| | (4-1BB)-(G₄S)₃-VL(4-1BB) | | |
| H2 | VH(4-1BB)-CH1-CH2-CH3-(G₄S)₃-VH(TNFR2)- | SEQ ID NO: 35 | IgG1 subtype |
| | (G₄S)₃-VL(TNFR2) | | |
| H3 | VH(4-1BB)-CH1-CH2-CH3 | SEQ ID NO: 36 | IgG1 subtype |
| H4 | VH(TNFR2)-(G₄S)₃-VL(TNFR2)-(G₄S)₃-VH | SEQ ID NO: 37 | IgG1 subtype |
| | (4-1BB)-CH1-CH2-CH3 | | |
| H5 | VH(TNFR2)-CH1-CH2-CH3-(G₄S)₃-VH(4-1BB)- | SEQ ID NO: 38 | IgG1 subtype |
| | (G₄S)₃-VL(4-1BB) | | |
| H6 | VH(TNFR2)-CH1-CH2-CH3 | SEQ ID NO: 39 | IgG1 subtype |
| H7 | VH(4-1BB)-(G₄S)₃-VL(4-1BB)-(G₄S)₃- | SEQ ID NO: 40 | IgG1 subtype |
| | VH(TNFR2)-CH1-CH2-CH3 | | |
| H8 | VH(4-1BB)-CH1-CH2-CH3-(G₄S)₃- | SEQ ID NO: 41 | IgG1 subtype |
| | VHH(TNFR2) | | |
| H9 | VHH(TNFR2)-(G₄S)₃-VH(4-1BB)-CH1-CH2- | SEQ ID NO: 42 | IgG1 subtype |
| | CH3 | | |
| H10 | VH(TNFR2)-CH1-CH2-CH3-(G₄S)₃-VH(4-1BB)- | SEQ ID NO: 43 | IgG4 subtype |
| | (G₄S)₃-VL(4-1BB) | | |
| H11 | VH(4-1BB)-CH1-CH2-CH3-(G₄S)₃- | SEQ ID NO: 44 | IgG4 subtype |
| | VHH(TNFR2) | | |
| H12 | VHH(TNFR2)-(G₄S)₃-VH(4-1BB)-CH1-CH2- | SEQ ID NO: 45 | IgG4 subtype |
| | CH3 | | |

According to the peptide chain combination in Table 2, the bispecific antibody sequences shown in Table 3 were designed, wherein antibody O contained only two peptide chains (i.e., one first peptide chain and one second peptide chain), and other antibodies except antibody O contained four peptide chains (i.e., two identical first peptide chains and two identical second peptide chains).

TABLE 3
Bispecific antibody sequences

| | Amino acid sequence of | Amino acid sequence of |
|---|---|---|
| Antibodies | the first peptide chain | the second peptide chain |
| Antibody A | SEQ ID NO: 26 | SEQ ID NO: 35 |
| Antibody B | SEQ ID NO: 27 | SEQ ID NO: 36 |
| Antibody C | SEQ ID NO: 26 | SEQ ID NO: 37 |
| Antibody D | SEQ ID NO: 28 | SEQ ID NO: 36 |
| Antibody E1 | SEQ ID NO: 29 | SEQ ID NO: 38 |
| Antibody E2 | SEQ ID NO: 29 | SEQ ID NO: 43 |
| Antibody F | SEQ ID NO: 30 | SEQ ID NO: 39 |
| Antibody G | SEQ ID NO: 29 | SEQ ID NO: 40 |
| Antibody H | SEQ ID NO: 31 | SEQ ID NO: 39 |
| Antibody J1 | SEQ ID NO: 26 | SEQ ID NO: 41 |
| Antibody J2 | SEQ ID NO: 26 | SEQ ID NO: 44 |
| Antibody K | SEQ ID NO: 32 | SEQ ID NO: 36 |
| Antibody M1 | SEQ ID NO: 26 | SEQ ID NO: 42 |
| Antibody M2 | SEQ ID NO: 26 | SEQ ID NO: 45 |
| Antibody N | SEQ ID NO: 33 | SEQ ID NO: 36 |
| Antibody O | SEQ ID NO: 34 | SEQ ID NO: 34 |

Each chain of the bispecific antibody with different structures designed above was gene synthesized, and then the antibody fragment was inserted into the PCDNA3.1 vector using molecular cloning technology to construct a mammalian cell expression plasmid, which was introduced into the host cell line CHO cells using liposome transfection, and the fermentation supernatant was obtained using cell Fed-batch. The fermentation supernatant was purified by affinity chromatography, ion exchange chromatography and a series of other steps to finally purify the constructed antibody. The purified antibody was tested for expression, purity, SDS-PAGE, etc. to confirm the bispecific antibody.

### Example 2 Detection of the Binding Activity of Antibodies to TNFR2 Protein by ELISA

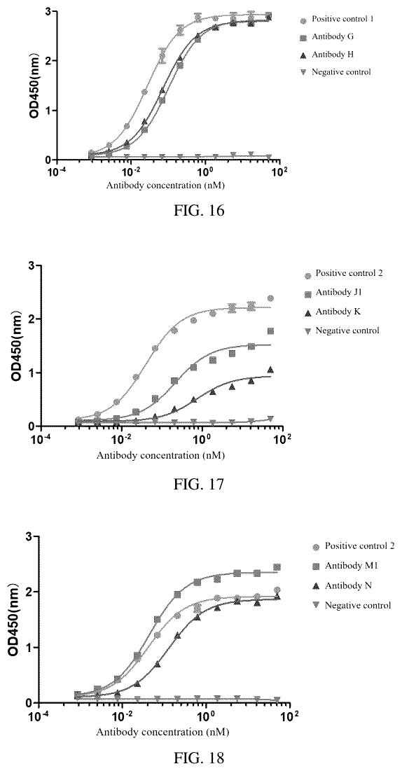

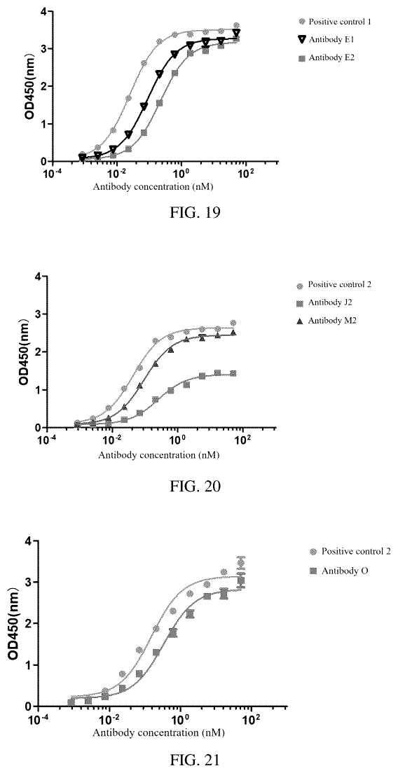

Human-TNFR2-His (manufacturer: SINO, CAT: 10417-H08H, LOT: LC15NO0412) was diluted to 0.1 μg/mL with a coating solution (1×PBS, pH7.4), and coated in 96-well ELISA plate at 100 L/well overnight at 4° C. The coating solution was poured off. Each well was washed with 1×PBST at 300 μL, washed 4 times with a plate washer, and patted dry on a flat paper. It was blocked with 3% skim milk powder at 300 μL/well, and incubated at 37° C. for 1 h. The blocking solution was poured off, washed 4 times with a plate washer, and patted dry on a flat paper. Negative control and positive control were set up. The negative control was commercially available trastuzumab for injection (Herceptin). The variable region of the antibody sequence of positive control 1 consists of SEQ ID NO: 5 and SEQ ID NO: 1, with the addition of the constant region of human IgG1 (SEQ ID NO: 46 and SEQ ID NO: 47); the variable region of the antibody sequence of positive control 2 consists of SEQ ID NO: 11. The positive control and antibody were diluted to 10 μg/mL with 3% skim milk powder as the starting concentration, and then diluted by 3-fold gradient (11 gradients in total), and a blank well was set up and only the diluent was added. It was incubated at 37° C. for 1 h at 100 μL/well. The liquid in the well was discarded, washed 4 times with a plate washer, and patted dry on a flat paper. Goat anti-human IgG Fc was diluted with 3% skim milk powder at 1:10000, and incubated at 37° C. for 1 h at 100 μL/well. It was washed 6 times with a plate washer, and patted dry on a flat paper. TMB color developing solution was added at 100 μL/well, and wrapped with aluminum foil. Color development was performed at 37° C. in the dark for 8 min. Termination solution 1M HCl was added to terminate the color development reaction at 100 μL/well. Readings were taken at 450 nm on an ELISA reader. The ELISA results of the antibody molecules are shown in FIGS. 14-21, respectively, and the results show that the antibodies can bind to the human TNFR2 protein.

### Example 3 Detection of the Binding Activity of Antibodies to 4-1BB Protein by ELISA

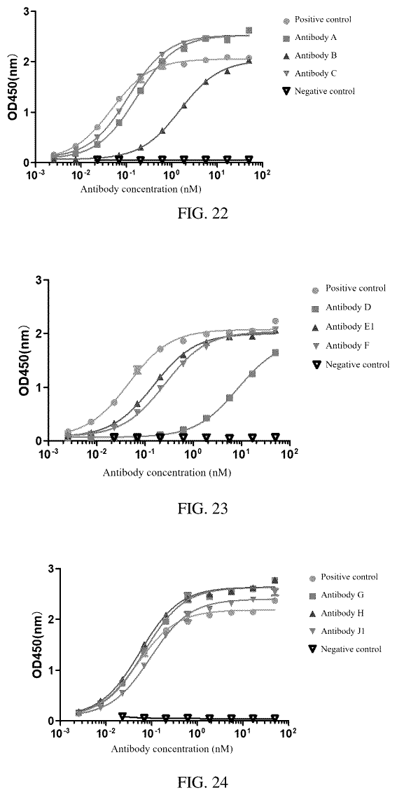

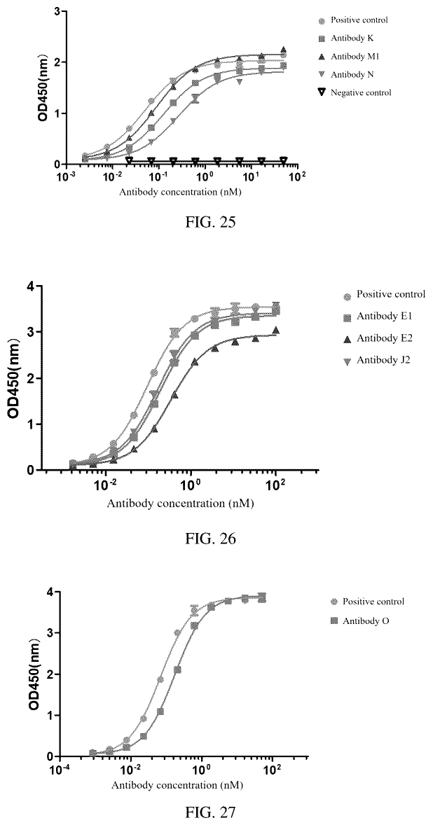

Human 4-1BB His (manufacturer: Acro, CAT: 41B-H5258, LOT: 198-2146F1-W6) was diluted to 0.2 μg/mL with a coating solution (1×PBS, pH7.4), and coated in 96-well ELISA plate at 100 μL/well overnight at 4° C. The coating solution was poured off. Each well was washed with 1×PBST at 300 μL, washed 4 times with a plate washer, and patted dry on a flat paper. It was blocked with 3% skim milk powder at 300 μL/well, and incubated at 37° C. for 1 h. The blocking solution was poured off, washed 4 times with a plate washer, and patted dry on a flat paper. Negative control and positive control were set up. The negative control was commercially available trastuzumab for injection (Herceptin). The variable region of the antibody sequence of the positive control consists of SEQ ID NO: 15 and SEQ ID NO: 19, with the addition of the constant region of human IgG1 (SEQ ID NO: 46 and SEQ ID NO: 47). The positive control and antibody were diluted to 10 μg/mL with 3% skim milk powder as the starting concentration, and then diluted by 3-fold gradient (11 gradients in total), and a blank well was set up and only the diluent was added. It was incubated at 37° C. for 1 h at 100 μL/well. The liquid in the well was discarded, washed 4 times with a plate washer, and patted dry on a flat paper. Goat anti-human IgG Fc was diluted with 3% skim milk powder at 1:20000, and incubated at 37° C. for 1 h at 100 μL/well. It was washed 6 times with a plate washer, and patted dry on a flat paper. TMB color developing solution was added at 100 μL/well, and wrapped with aluminum foil. Color development was performed at 37° C. in the dark for 8 min. Termination solution 1M HCl was added to terminate the color development reaction at 100 μL/well. Readings were taken at 450 nm on an ELISA reader. The ELISA results of the antibody molecules are shown in FIGS. 22-27, respectively, and the results show that the antibodies can bind to the human 4-1BB protein.

### Example 4 Binding Activity at Both Ends of the Constructed Antibodies

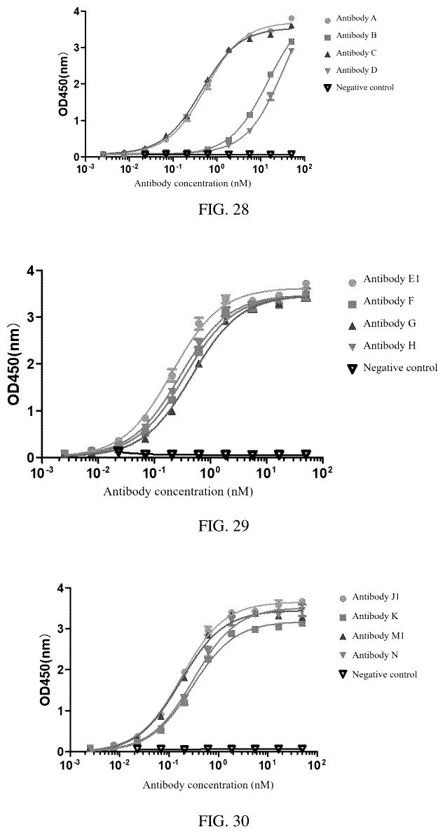

Human 4-1BB Fc (manufacturer: Acro, CAT: 41B-H5258, LOT: 198-2146F1-W6) was diluted to 0.3 μg/mL with a coating solution (1×PBS, pH7.4) and coated into a 96-well ELISA plate at 100 L/well at 4° C. overnight. The coating solution was poured off. Each well was washed with 1×PBST at 300 μL, washed 4 times with a plate washer, and patted dry on a flat sheet. It was blocked with 3% skim milk powder at 300 μL/well, and incubated at 37° C. for 1 h. The blocking solution was poured off, washed 4 times with a plate washer, and patted dry on a flat sheet. A negative control was set up, which was commercially available trastuzumab for injection (Herceptin). Starting at 50 nM, which was used as the initial concentration for 3-fold dilution, a total of 10 gradients were diluted, and a blank well was set up and only the diluent was added. It was incubated at 37° C. for 1 h at 100 μL/well. The liquid in the well was discarded, washed 4 times with a plate washer, and patted dry on a flat paper. Human-TNFR2-His (manufacturer: Sino, CAT: 10417-H08H, LOT: LC15NO0412) was diluted to 0.3 μg/mL, 100 μL was added to each well, and incubated at room temperature for 1 h. The plate was washed 3 times with PBST, and then the HRP-labeled his antibody was diluted with sample diluent at 1:5000, 100 μ L was added to each well, and incubated at room temperature for 1 h. It was washed 6 times with a plate washer, and patted dry on a flat paper. TMB color developing solution was added at 100 μL/well, and wrapped with aluminum foil. Color development was performed at 37° C. in the dark for 8 min. Termination solution 1M HCl was added to terminate the color development reaction at 100 μL/well. Readings were taken at 450 nm on an ELISA reader. The ELISA results of the antibody molecules are shown in FIGS. 28-30, respectively, and the results show that the antibodies have binding activity at both ends.

### Example 5 Binding Activity at Both Ends of the Constructed Antibodies

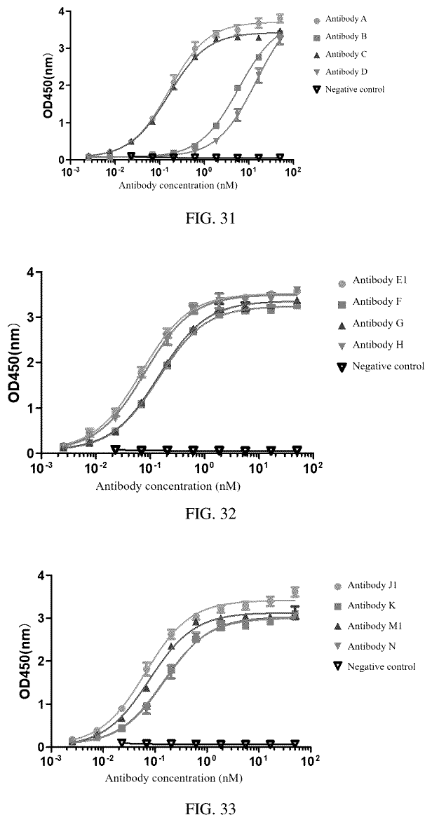

Human TNFR2 mFc (manufacturer: Kaixia, CAT: TN-HM3R2, LOT: 031202) was diluted to 0.3 μg/mL with a coating solution (1×PBS, pH7.4) and coated into a 96-well ELISA plate at 100 μL/well at 4° C. overnight. The coating solution was poured off. Each well was washed with 1×PBST at 300 μL, washed 4 times with a plate washer, and patted dry on a flat paper. It was blocked with 3% skim milk powder at 300 μL/well, and incubated at 37° C. for 1 h. The blocking solution was poured off, washed 4 times with a plate washer, and patted dry on a flat paper. A negative control was set up, which was commercially available trastuzumab for injection (Herceptin). Starting at 50 nM, which was used as the initial concentration for 3-fold dilution, a total of 10 gradients were diluted, and a blank well was set up and only the diluent was added. It was incubated at 37° C. for 1 h at 100 L/well. The liquid in the well was discarded, washed 4 times with a plate washer, and patted dry on a flat paper. Human 4-1BB His (manufacturer: Acro, CAT: 41B-H5258, LOT: 198-2146F1-W6) was diluted to 0.3 μg/mL, 100 μL was added to each well, and incubated at room temperature for 1 h. The plate was washed 3 times with PBST, and then the HRP-labeled his antibody was diluted with sample diluent at 1:5000, 100 μ L was added to each well, and incubated at room temperature for 1 h. It was washed 6 times with a plate washer, and patted dry on a flat paper. TMB color developing solution was added at 100 μL/well, and wrapped with aluminum foil. Color development was performed at 37° C. in the dark for 8 min. Termination solution 1M HCl was added to terminate the color development reaction at 100 μL/well. Readings were taken at 450 nm on an ELISA reader. The ELISA results of the antibody molecules are shown in FIGS. 31-33, respectively, and the results show that the antibodies have binding activity at both ends.

### Example 6 Detection of the Affinity of Antibodies to TNFR2 Protein by BLI Method

The variable region of the antibody sequence of positive control 1 consists of SEQ ID NO: 5 and SEQ ID NO: 1, with the addition of the constant region of human IgG1 (SEQ ID NO: 46 and SEQ ID NO: 47); the variable region of the antibody sequence of positive control 2 consists of SEQ ID NO: 11. The AHC sensor was soaked in 0.02% PBST (0.02% Tween 20, pH 7.4, 1×PBS) as a buffer for 600 s to remove the sucrose covering the sensor surface. The AHC sensor was equilibrated with 0.02% PBST (0.02% Tween 20, pH 7.4, 1×PBS) as a buffer for 60 s, the antibody in the sample plate was solidified for 300 s, and the secondary equilibration buffer was used for 180 s. 100 nM human-TNFR2-His (manufacturer: SINO, CAT: 10417-H08H, LOT: LC15NO0412) protein was bound to the antibody for 300 s and then dissociated for 600 s. After dissociation, 10 mM glycine (pH 2.0) was used as a regeneration buffer for 30 s. The sensor was regenerated with 10 mM glycine (pH 2.0). The affinity results of the antibody molecules are shown in Table 4. The results show that the antibodies all have affinity for TNFR2 protein.

TABLE 4
Affinity of antibodies to TNFR2 protein

| | KD(M) | kon(1/Ms) | kdis(1/s) |
|---|---|---|---|
| Positive control 1 | 7.24 × 10⁻¹¹ | 2.57 × 10⁵ | 1.86 × 10⁻⁵ |
| Antibody E1 | 2.07 × 10⁻¹⁰ | 3.89 × 10⁵ | 8.04 × 10⁻⁵ |
| Antibody H | 2.41 × 10⁻¹⁰ | 2.76 × 10⁵ | 6.64 × 10⁻⁵ |
| Positive control 2 | 1.61 × 10⁻⁹ | 2.66 × 10⁵ | 4.27 × 10⁻⁴ |
| Antibody J1 | 1.82 × 10⁻⁹ | 2.17 × 10⁵ | 3.94 × 10⁻⁴ |
| Antibody M1 | 1.28 × 10⁻⁹ | 3.08 × 10⁵ | 3.94 × 10⁻⁴ |
| Antibody O | 3.11 × 10⁻⁸ | 3.78 × 10⁴ | 1.18 × 10⁻³ |

### Example 7 Detection of the Affinity of Antibodies to 4-1BB Protein by BLI Method

The variable region of the antibody sequence of the positive control consists of SEQ ID NO: 15 and SEQ ID NO: 19, with the addition of the constant region of human IgG1 (SEQ ID NO: 46 and SEQ ID NO: 47). The AHC sensor was soaked in 0.02% PBST (0.02% Tween 20, pH 7.4, 1× PBS) as a buffer for 600 s to remove the sucrose covering the sensor surface. The AHC sensor was equilibrated with 0.02% PBST (0.02% Tween 20, pH 7.4, 1× PBS) as a buffer for 60 s, the antibody in the sample plate was solidified for 300 s, and the secondary equilibration buffer was used for 180 s. 100 nM human 4-1BB His (manufacturer: Acro, CAT: 41B-H5258, LOT: 198-2146F1-W6) protein was bound to the antibody for 300 s and then dissociated for 600 s. After dissociation, 10 mM glycine (pH 2.0) was used as a regeneration buffer for 30 s. The sensor was regenerated with 10 mM glycine (pH 2.0). The affinity results of the antibody molecules are shown in Table 5, and the results show that the antibodies all have affinity for the 4-1BB protein.

TABLE 5
Affinity of antibodies to TNFR2 protein

| | KD(M) | kon(1/Ms) | kdis(1/s) |
|---|---|---|---|
| Positive control | 1.42 × 10⁻⁸ | 3.91 × 10⁵ | 5.53 × 10⁻³ |
| Antibody E1 | 1.81 × 10⁻⁸ | 3.02 × 10⁵ | 5.46 × 10⁻³ |
| Antibody H | 6.42 × 10⁻⁹ | 6.77 × 10⁵ | 4.35 × 10⁻⁵ |
| Antibody J1 | 1.22 × 10⁻⁸ | 5.03 × 10⁵ | 6.16 × 10⁻³ |
| Antibody M1 | 1.75 × 10⁻⁸ | 3.69 × 10⁵ | 6.47 × 10⁻³ |
| Antibody O | 1.35 × 10⁻⁷ | 5.31 × 10⁴ | 7.15 × 10⁻³ |

### Example 8 Assay of HEK-293/NFκB-Luci/4-1BB Cell Activation

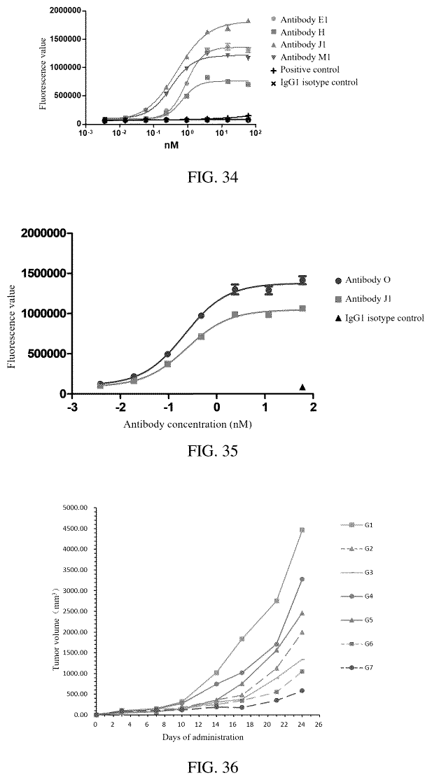

The variable region of the antibody sequence of the positive control consists of SEQ ID NO: 15 and SEQ ID NO: 19, with the addition of the constant region of human IgG1 (SEQ ID NO: 46 and SEQ ID NO: 47). The actual starting and ending concentration of the antibody was 60 nM with 100 μL/well, 4-fold dilution, and 8 gradients. HEK-293/NFκB-Luci/4-1BB effector cells (from the cell bank of Shenghe (China) Biopharmaceutical Co., Ltd.) were added to the plate with a cell density of 3×10⁴/well at 40 μL/well; CHOK1-TNFR2 target cells were added to the plate with a cell density of 1×10⁴/well at 40 μL/well; diluted antibodies were added to the plate at 20 μL/well; after the addition of samples, the 96-well full black plate was placed in a 37° C. incubator for 20 h. Lumiescence fluorescent agent was added at 100 μL/well. The results of the test are shown in FIG. 34. The results show that antibodies E1, H, J1 and M1 all have an activating effect on HEK-293/NFκB-Luci/4-1BB effector cells.

### Example 9 Assay of HEK-293/NFκB-Luci/4-1BB Cell Activation

The actual starting and ending concentration of the antibody was 60 nM with 100 μL/well, 5-fold dilution, and 7 gradients. HEK-293/NFκB-Luci/4-1BB effector cells (from the cell bank of Shenghe (China) Biopharmaceutical Co., Ltd.) were added to the plate with a cell density of 3×10⁴/well at 40 μL/well; CHOK1-TNFR2 target cells were added to the plate with a cell density of 1×10⁴/well at 40 μL/well; diluted antibodies were added to the plate at 20 μL/well; after the addition of the sample, the 96-well full black plate was placed in a 37° C. incubator for 20 h. Lumiescence fluorescent agent was added at 100 μL/well. The results of the machine detection are shown in FIG. 35, which shows that both antibody O and antibody J1 have an activating effect on HEK-293/NFκB-Luci/4-1BB effector cells.

### Example 10 Pharmacodynamic Study of Each Test Substance in the Subcutaneously Transplanted Mouse Colon Cancer Cell Line MC38 Tumor Model in C57BL/6-h4-1BB/hTNFR2 Mice

Female C57BL/6-h4-1BB/hTNFR2 mice aged 6-7 weeks were subcutaneously inoculated with MC38 tumor cells (from the cell bank of Shenghe (China) Biopharmaceutical Co., Ltd.) and randomly divided into seven groups after the tumor volume reached about 100±50 mm³. The groups included: (1) G1: PBS group; (2) G2: control antibody group, the sequence variable region consists of SEQ ID NO: 15 and SEQ ID NO: 19, with the addition of the constant region of human IgG1 (SEQ ID NO: 46 and SEQ ID NO: 47); (3) G3: antibody E1 group; (4) G4: antibody E2 group; (5) G5: antibody J2 group; (6) G6: antibody M2 group; and (7) G7: antibody O group. The negative control group was intratumorally administered with PBS, and the samples of the other groups were intratumorally administered with 10 mg/kg. The drug administration frequency was twice a week for 4 consecutive weeks, for a total of 8 times; the tumor volume and body weight of the mice were measured every other day, and the tumor volume was calculated according to a·b²/2 (a is the long diameter and b is the short diameter). The experimental design was shown in Table 6.

TABLE 6
Experimental design

| | | | Inoculation | | | | Dosing |
|---|---|---|---|---|---|---|---|
| | Mice | | amount | | Dose | Route of | frequency and |
| Group | (pcs) | Cell | (pieces/unit) | Test drug | (mg/kg) | administration | observation |
| G1 | 4 | MC38 | 5 × 10⁵ | / | / | Intraperitoneal | 2 times a week |
| | | | | | | injection | |
| G2 | 4 | MC38 | 5 × 10⁵ | Control | 10 | Intraperitoneal | 2 times a week |
| | | | | antibody | | injection | |
| G3 | 4 | MC38 | 5 × 10⁵ | Antibody E1 | 10 | Intraperitoneal | 2 times a week |
| | | | | | | injection | |
| G4 | 5 | MC38 | 5 × 10⁵ | Antibody E2 | 10 | Intraperitoneal | 2 times a week |
| | | | | | | injection | |
| G5 | 4 | MC38 | 5 × 10⁵ | Antibody J2 | 10 | Intraperitoneal | 2 times a week |
| | | | | | | injection | |
| G6 | 5 | MC38 | 5 × 10⁵ | Antibody M2 | 10 | Intraperitoneal | 2 times a week |
| | | | | | | injection | |
| G7 | 4 | MC38 | 5 × 10⁵ | Antibody O | 10 | Intraperitoneal | 2 times a week |
| | | | | | | injection | |

The results are shown in FIG. 36, and antibody M2 and antibody O have significant anti-tumor effects.

### Example 11 Sequence Optimization of Antibody O

The peptide chains in Table 7 can be formed by connecting the sequence of the TNFR2 antibody and the sequence of the 4-1BB antibody through a linker in a certain order. Among them, in this embodiment, the linker used in the bispecific antibody was 3 GGGGS repeats (i.e., GGGGSGGGGSGGGGS, hereinafter abbreviated as (G₄S)₃) or 4 GGGGS repeats (i.e., GGGGSGGGGSGGGGSGGGGS, hereinafter abbreviated as ((G₄S)₄), or SPNSASHSGSAPQTSSAPGSQ or GGGSAAA. The introduction of cysteine refers to the introduction of one cysteine in each of VH (4-1BB) and VL (4-1BB). The CL used in this embodiment was a kappa (κ) type, and the sequence was as shown in SEQ ID NO: 25.

TABLE 7
Peptide chain sequence list

| Peptide | | Amino acid | | |
|---|---|---|---|---|
| chain | Structural Order | sequence | Remark | Subtype |
| H13 | VHH(TNFR2)-Fc-(G₄S)₃-VH(4-1BB)-(G₄S)₃- | SEQ ID NO: 48 | Cysteine | IgG1 |
| | VL(4-1BB) | | introduced | subtype |
| H14 | VHH(TNFR2)-Fc-(G₄S)₃-VH(4-1BB)-(G₄S)₃- | SEQ ID NO: 49 | / | IgG1 |
| | VL(4-1BB) | | | subtype |
| H15 | VHH(TNFR2)-Fc-(G₄S)₃-VH(4-1BB)-(G₄S)₄- | SEQ ID NO: 50 | Cysteine | IgG1 |
| | VL(4-1BB) | | introduced | subtype |
| H16 | VHH(TNFR2)-Fc-(G₄S)₃-VH(4-1BB)-(G₄S)₄- | SEQ ID NO: 51 | / | IgG1 |
| | VL(4-1BB) | | | subtype |
| H17 | VHH(TNFR2)-Fc-(G₄S)₄-VH(4-1BB)-(G₄S)₃- | SEQ ID NO: 52 | / | IgG1 |
| | VL(4-1BB) | | | subtype |
| H18 | VHH(TNFR2)-Fc-(G₄S)₄-VH(4-1BB)- | SEQ ID NO: 53 | / | IgG1 |
| | SPNSASHSGSAPQTSSAPGSQ-VL(4-1BB) | | | subtype |
| H19 | VHH(TNFR2)-Fc-(G₄S)₄-VH(4-1BB)- | SEQ ID NO: 54 | / | IgG1 |
| | GGGSAAA-VL(4-1BB) | | | subtype |
| H20 | VHH(TNFR2)-Fc-(G₄S)₄-VH(4-1BB)-(G₄S)₄- | SEQ ID NO: 55 | Cysteine | IgG1 |
| | VL(4-1BB) | | introduced | subtype |

According to the peptide chain combinations in Table 7, the bispecific antibody sequences shown in Table 8 were designed, and the antibodies all contained only two peptide chains (ie, one first peptide chain and one second peptide chain).

TABLE 8
Bispecific antibody sequences

| | Amino acid sequence of | Amino acid sequence of |
|---|---|---|
| Antibodies | the first peptide chain | the second peptide chain |
| Antibody O1 | SEQ ID NO: 48 | SEQ ID NO: 48 |
| Antibody O2 | SEQ ID NO: 49 | SEQ ID NO: 49 |
| Antibody O3 | SEQ ID NO: 50 | SEQ ID NO: 50 |
| Antibody O4 | SEQ ID NO: 51 | SEQ ID NO: 51 |
| Antibody O5 | SEQ ID NO: 52 | SEQ ID NO: 52 |
| Antibody O6 | SEQ ID NO: 53 | SEQ ID NO: 53 |
| Antibody O7 | SEQ ID NO: 54 | SEQ ID NO: 54 |
| Antibody O8 | SEQ ID NO: 55 | SEQ ID NO: 55 |

Each chain of the bispecific antibody with different structures designed above was gene synthesized, and then the antibody fragment was inserted into the PCDNA3.1 vector using molecular cloning technology to construct a mammalian cell expression plasmid, which was introduced into the host cell line CHO cells using liposome transfection, and the fermentation supernatant was obtained using cell Fed-batch. The fermentation supernatant was purified by affinity chromatography, ion exchange chromatography and a series of other steps to finally purify the constructed antibody. The purified antibody was tested for expression, purity, SDS-PAGE, etc. to confirm the bispecific antibody.

### Example 12 Detection of the Binding Activity of Antibodies to TNFR2 Protein by ELISA

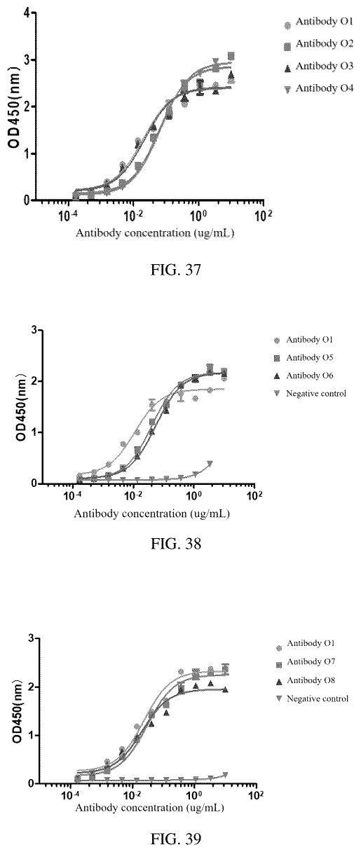

Human-TNFR2-His (manufacturer: SINO, CAT: 10417-H08H, LOT: LC15SE2206) was diluted to 0.1 μg/mL with a coating solution (1×PBS, pH7.4), and coated in 96-well ELISA plate at 100 μL/well at 4° C. overnight. The coating solution was poured off, the plate was washed with 1×PBST at 300 μL/well, washed 4 times with a plate washer, and patted dry on a flat paper. It was blocked with 3% skim milk powder at 300 μL/well, and incubated at 37° C. for 1 h. The blocking solution was poured off, washed 4 times with a plate washer, and patted dry on a flat paper. Negative control and positive control were set up. The negative control was the commercially available trastuzumab for injection (Herceptin), and the positive control was antibody 01. The negative control, positive control and the antibody were diluted to 10 μg/mL with 3% skim milk powder as the starting concentration, and then diluted by 3-fold gradient (11 gradients in total). A blank well was set up and only the diluent was added at 100 μL/well, and incubated at 37° C. for 1 h. The liquid in the well was discarded, washed 4 times with a plate washer, and patted dry on a flat paper. Goat anti-human IgG Fc was diluted with 3% skim milk powder at 1:20000 at 100 μL/well, and incubated at 37° C. for 1 h. It was washed 6 times with a plate washer, and patted dry on a flat paper. TMB color developing solution was added at 100 μL/well, and wrapped with aluminum foil. Color development was performed at 37° C. in the dark for 8 min. Termination solution 1M HCl was added at 100 μL/well to terminate the color development reaction. Readings were taken at 450 nm on an ELISA reader. The ELISA results of the antibody molecules are shown in FIGS. 37-39, respectively, and the results show that the antibodies can bind to the human TNFR2 protein.

### Example 13 Detection of the Binding Activity of Antibodies to 4-1BB Protein by ELISA

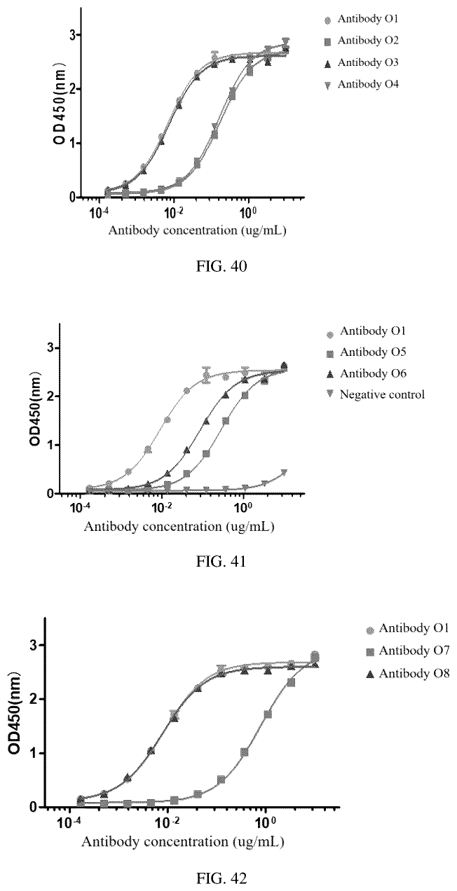

Human 4-1BB His (manufacturer: Acro, CAT: 41B-H5258, LOT: 2407c-214FF1-YC) was diluted to 0.2 μg/mL with a coating solution (1×PBS, pH7.4), and coated into a 96-well ELISA plate at 100 μL/well at 4° C. overnight. The coating solution was poured off, washed well with 1×PBST at 300 μL/well, washed 4 times with a plate washer, and patted dry on a flat paper. It was blocked with 3% skim milk powder at 300 μL/well, and incubated at 37° C. for 1 h. The blocking solution was poured off, washed 4 times with a plate washer, and patted dry on a flat paper. Negative control and positive control were set up. The negative control was the commercially available trastuzumab for injection (Herceptin), and the positive control was antibody 01. The negative control, positive control and the antibody were diluted to 10 μg/mL with 3% skim milk powder as the starting concentration, and then diluted by 3-fold gradient (11 gradients in total). A blank well was set up and only the diluent was added at 100 μL/well, and incubated at 37° C. for 1 h. The liquid in the well was discarded, washed 4 times with a plate washer, and patted dry on a flat paper. Goat anti-human IgG was diluted with 3% skim milk powder at 1:20000 at 100 μL/well, and incubated at 37° C. for 1 h. It was washed 6 times with a plate washer, and patted dry on a flat paper. TMB color developing solution was added at 100 μL/well, and wrapped with aluminum foil. Color development was performed at 37° C. in the dark for 8 min. Termination solution 1M HCl was added to terminate the color development reaction at 100 μL/well. Readings were taken at 450 nm on an ELISA reader. The ELISA results of the antibody molecules are shown in FIGS. 40-42, respectively. The antibodies can bind to human 4-1BB protein.

The protection content of the present invention is not limited to the above embodiments. Without departing from the spirit and scope of the inventive concept, changes and advantages that can be thought of by those skilled in the art are included in the present invention and are protected by the attached claims.

1. A bispecific antibody that specifically binds to TNFR2 and 4-1BB, the bispecific antibody comprising:
(a) a first antibody or antigen-binding fragment thereof that specifically binds to a first antigen; and
(b) a second antibody or antigen-binding fragment thereof that specifically binds to a second antigen; wherein:
the first antigen is TNFR2, and the second antigen is 4-1BB; or, the first antigen is 4-1BB, and the second antigen is TNFR2.

2. The bispecific antibody of claim 1, wherein the first antibody or antigen-binding fragment thereof comprises a heavy chain and a light chain; and the second antibody or antigen-binding fragment thereof comprises scFv or VHH.

3. The bispecific antibody of claim 2, wherein the heavy chain of the first antibody or antigen-binding fragment thereof comprises a heavy chain variable region and a heavy chain constant region, and the light chain comprises a light chain variable region and a light chain constant region.

4. The bispecific antibody of claim 3, wherein the heavy chain variable region of one heavy chain and the light chain variable region of one light chain of the first antibody form an antigen-binding site, and the heavy chain variable region of another heavy chain and the light chain variable region of another light chain form an antigen-binding site.

5. The bispecific antibody of claim 4, comprising one first antibody or antigen-binding fragment thereof and one or more scFv or VHH.

6. The bispecific antibody of claim 5, comprising one first antibody or antigen-binding fragment thereof and one scFv or one VHH, wherein the scFv or VHH is connected to the N-terminus or C-terminus of the heavy chain or light chain of the first antibody or antigen-binding fragment thereof.

7. The bispecific antibody of claim 5, comprising one first antibody or antigen-binding fragment thereof and two scFvs or two VHHs.

8. The bispecific antibody of claim 7, wherein the two scFvs or two VHHs are respectively connected to the N-terminus of the two heavy chains or the two light chains of the first antibody or antigen-binding fragment thereof.

9. The bispecific antibody of claim 7, wherein the two scFvs or two VHHs are respectively connected to the C-terminus of the two heavy chains or the two light chains of the first antibody or antigen-binding fragment thereof.

10. The bispecific antibody of claim 8, further comprising two first polypeptide chains and two second polypeptide chains, wherein for each of the polypeptide chains:
(a) the first polypeptide chain independently comprises the light chain of the first antibody or antigen-binding fragment thereof; and
(b) the second polypeptide chain independently comprises the heavy chain of the first antibody or antigen-binding fragment thereof and the scFv or VHH.

11. The bispecific antibody of claim 8, further comprising two first polypeptide chains and two second polypeptide chains, wherein for each of the polypeptide chains:
(a) the first polypeptide chain independently comprises the light chain of the first antibody or antigen-binding fragment thereof and the scFv or VHH; and
(b) the second polypeptide chain independently comprises the heavy chain of the first antibody or antigen-binding fragment thereof.

12. The bispecific antibody of claim 10, wherein the two first polypeptide chains are identical or different, and/or the two second polypeptide chains are identical or different.

13. The bispecific antibody of claim 9, wherein the Fab region of the first antibody or antigen-binding fragment thereof is replaced by a VHH that specifically binds to TNFR2.

14. The bispecific antibody of claim 13, further comprising two polypeptide chains, wherein each of the polypeptide chains independently comprises the first antibody or antigen-binding fragment thereof and the scFv.

15. The bispecific antibody of claim 14, wherein the two polypeptide chains are identical or different.

16. The bispecific antibody of claim 15, wherein the heavy chain variable region and the light chain variable region of the scFv are connected by a linker L1.

17. The bispecific antibody of claim 15, wherein the scFv is connected to the N-terminus or C-terminus of the heavy chain or light chain of the first antibody or antigen-binding fragment thereof through a linker L2.

18. The bispecific antibody of claim 12, wherein the VHH as the second antibody or antigen-binding fragment thereof is connected to the N-terminus or C-terminus of the heavy chain or light chain of the first antibody or antigen-binding fragment thereof by a linker L2.

19. The bispecific antibody of claim 18, wherein the linker L1 and the linker L2 are identical or different.

20. The bispecific antibody of claim 19, wherein the linker L1 and/or linker L2 has an amino acid sequence as shown in (G₄S)ₓ, where x is an integer selected from 1 to 6; preferably, the linker L1 and/or linker L2 is (G₄S)₂, (G₄S)₃ or (G₄S)₄.

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)
